(19) United States
(12) Patent Application Publication
(10) Pub. No.: US 2018/0369337 A1
(43) Pub. Date: Dec. 27, 2018

(54) Methods of Using Interleukin-10 for Treating Diseases and Disorders

(71) Applicant: ARMO BioSciences, Inc., Redwood City, CA (US)

(72) Inventors: John Brian Mumm, Los Altos Hills, CA (US); Ivan Ho Chan, Millbrae, CA (US)

(21) Appl. No.: 16/061,583

(22) PCT Filed: Dec. 28, 2016
(86) PCT No.: PCT/US16/68945

§ 371 Date: Jun. 12, 2018

Related U.S. Application Data
(60) Provisional application No. 62/275,127, filed on Jan. 5, 2016.

(51) Int. Cl. A61K 38/20; A61P 35/02; A61K 47/60

(52) U.S. Cl. CPC A61K 38/2066; A61K 38/208; A61K 45/06; A61K 47/60; A61P 35/02

(57) ABSTRACT

Methods of treating subjects having a cancer-related disease, disorder, or condition, or preventing the occurrence of such a disease, disorder or condition, via the administration of a PEG-IL-10 in combination with an IL-12 agent are provided.

### CROSS-REFERENCE TO RELATED APPLICATION

This application claims priority benefit of US Provisional Application Ser. No. 62/275,127, filed Jan. 5, 2016, which application is incoroproated by reference herein in its entirety.

### FIELD OF THE INVENTION

This invention relates to methods of using a PEG-IL-10 in combination with other agents in the treatment or prevention of a diverse array of diseases and disorders, including cancers and immune-related disorders.

### INTRODUCTION

The cytokine interleukin-10 (IL-10) is a pleiotropic cytokine that regulates multiple immune responses through actions on T cells, B cells, macrophages, and antigen presenting cells (APC). IL-10 can suppress immune responses by inhibiting expression of IL-1α, IL-1β, IL-6, IL-8, TNFα, GM-CSF and G-CSF in activated monocytes and activated macrophages, and it also suppresses IFN-γ production by NK cells. Although IL-10 is predominantly expressed in macrophages, expression has also been detected in activated T cells, B cells, mast cells, and monocytes. In addition to suppressing immune responses, IL-10 exhibits immuno-stimulatory properties, including stimulating the proliferation of IL-2—and IL-4—treated thymocytes, enhancing the viability of B cells, and stimulating the expression of MHC class II.

Human IL-10 is a homodimer that becomes biologically inactive upon disruption of the non-covalent interactions between the two monomer subunits. Data obtained from the published crystal structure of IL-10 indicates that the functional dimer exhibits certain similarities to IFN-γ (Zdanov et al, (1995) Structure (Lond) 3:591-601).

As a result of its pleiotropic activity, IL-10 has been linked to a broad range of diseases, disorders and conditions, including inflammatory conditions, immune-related disorders, fibrotic disorders, metabolic disorders and cancer. Clinical and pre-clinical evaluations with IL-10 for a number of such diseases, disorders and conditions have solidified its therapeutic potential. Moreover, pegylated IL-10 has been shown to be more efficacious than non-pegylated IL-10 in certain therapeutic settings.

### SUMMARY

The present disclosure contemplates methods of using a PEG-IL-10 (e.g., rHuPEG-IL-10), and compositions thereof, in combination with an IL-12 agent (e.g., rHuIL-12), and compositions thereof, for the treatment and/or prevention of cancer-related diseases, disorders and conditions, and/or the symptoms thereof. The methods provide the opportunity for additive, and perhaps synergistic, effects in the treatment and/or prevention of the cancer-related diseases, disorders and conditions described herein. Moreover, such combination therapy may allow for reduction in the amounts and/or frequencies of administration of the PEG-IL-10 and/or the IL-12 agent in which it is combined, which can result in any adverse effects being minimized or obviated. The combination therapy encompasses co-administration when the PEG-IL-10 and IL-12 agent are administered separately (e.g., two distinct pharmaceutical compositions) or together (e.g., one pharmaceutical composition comprising both the PEG-IL-10 and the IL-12 agent).

As discussed in detail herein, IL-10 is deemed to be an anti-inflammatory and immuno-suppressive cytokine that inhibits the secretion of IFNγ, IL-12 and TNFα. It also inhibits antigen presentation and subsequent activation of CD4+ T cells and is thus widely considered to be a potent immune suppressive cytokine. In clinical studies involving various cancer patient populations, subcutaneous administration of PEG-IL-10 as monotherapy has yielded beneficial results.

In particular, recent evidence indicates that PEG-IL-10 exerts immunostimulatory effects in context of immunoncology (Infante, J. R., et al., ASCO Meeting Abstracts, 2015. 33(15_suppl): p. 3017). Though an understanding of the specific mechanism of this anti-tumor effect is not required to practice the present disclosure, the effect has been shown to require both CD8+ T cells and endogenous IFNγ (Mumm, J. B., et al., Cancer Cell, 2011. 20(6): p. 781-96; Emmerich, J., et al., Cancer Res, 2012. 72(14): p. 3570-81). Specifically, CD8+ T cell exposure to PEG-IL-10 leads to the potentiation of IFNγ, Granzyme B and Perforin secretion. The secretion of both IFNγ and Granzyme B are dependent upon T cell receptor engagement with cognate MHC I/antigen complexes (Chan, I. H., et al., J Interferon Cytokine Res, 2015).

Treatment of human cancer patients with PEG-rHuIL-10 leads to substantial monotherapy anti-tumor responses characterized by substantial increases in Granzyme B+ intratumoral CD8+ T cell infiltration. Concomitant with this activated CD8+ intratumoral T cell infiltrate is a reproducible increase in the serum cytokines IFNγ, IL-18, IL-7, IL-4, GM-CSF and the activated T cell marker FasL. In addition, treatment with PEG-rHuIL-10 decreases serum TGF-β. These cytokines are the hallmarks of broad spectrum immune activation.

Human IL-10 is a homodimer, and each monomer comprises 178 amino acids, the first 18 of which comprise a signal peptide. Unless otherwise indicated, reference herein to human IL-10 refers to the mature form that lacks the signal peptide, wherein each monomer comprises 160 amino acids (see, e.g., U.S. Pat. No. 6,217,857). As used herein, the term “PEG-IL-10” refers to pegylated human IL-10 and variants thereof that exhibit activity comparable to the activity of mature human PEG-IL-10, such as pegylated murine IL-10 and pegylated forms of other IL-10 orthologs.

Interleukin-12 (IL-12) is a pleiotropic cytokine naturally produced by macrophages, B-lymphoblastoid cells, dendritic cells, and neutrophils in response to antigenic stimulation. It is involved in the differentiation of naöve T cells into Thi cells, can stimulate the growth and function of T cells, and mediates enhancement of the cytotoxic activity of NK cells and CD8+ cytotoxic T lymphocytes. As discussed further herein, IL-12 also stimulates the production of IFNγ and TNFα from T cells and NK cells, and reduces IL-4 mediated suppression of IFNγ. IFNγ has been shown to coordinate natural mechanisms of anticancer defense (Jakobisiak, M. et al. (2013) Immunol Lett 90:103-22). Due, in part, to its potent stimulation of IFNγ production, IL-12 was initially thought to represent an ideal candidate for tumor immunotherapy. However, systemic administration of IL-12 during initial clinical studies yielded a very narrow therapeutic index and resulted in an unacceptable adverse effect profile. As a result, systemic administration of IL-12 as a monotherapy in the oncology setting was largely deemed unviable. (See, e.g., Teng, M. et al. (2015) Nature Medicine 21(7):719-29).

As used herein, the terms “IL-12”, “IL-12 polypeptide(s),” “IL-12-agent(s)”, “IL-12 molecule(s)” and the like are intended to be construed broadly and include, for example, human and non-human IL-12—related polypeptides, including homologs, variants (including muteins), and fragments thereof, as well as IL-12 polypeptides having, for example, a leader sequence (e.g., a signal peptide).

In particular embodiments, the present disclosure contemplates methods of treating or preventing a cancer-related disease, disorder or condition in a subject, comprising administering to the subject: a) a therapeutically effective amount of an IL-12 agent, and b) a therapeutically effective amount of a PEG-IL-10; wherein the amount of the PEG-IL-10 is sufficient to reduce the IL-12—associated toxicity to a level less than that observed with IL-12 monotherapy.

IL-12—associated toxicities include flu-like symptoms (e.g., headache, fever, chills, fatigue and arthromyalgia); hematologic toxicity, including neutropenia and thrombocytopenia; and hepatic toxicity, manifested by dose-dependent increases in transaminases, hyperbilirubinemia, and hypoalbuminemia. Other IL-12 associated adverse effects include inflammation of mucus membranes (e.g., oral mucositis, stomatitis and colitis), hypotension, renal impairment and gastrointestinal bleeding. These toxic effects have been associated with the secondary production of IFNγ and TNFα, as well as other cytokines (e.g., IP-10 and MIG). (See, e.g., Lasek, et al. (2014) Cancer Immunol Immunother 63:419-35; Xu, et al. Clinical and Developmental Immunology, volume 2010, Article ID 832454, 9 pp.); Cebon, J., et al., Cancer Immun, 2003. 3: p. 7).

In other embodiments, the present disclosure contemplates methods of treating or preventing a cancer-related disease, disorder or condition in a subject, comprising administering to the subject: a) a therapeutically effective amount of an IL-12 agent; and b) a therapeutically effective amount of a PEG-IL-10, wherein the amount of the PEG-IL-10 is sufficient to i) achieve a mean IL-10 serum trough concentration of at least 1.0 ng/mL, and ii) reduce the IL-12—associated toxicity to a level less than that observed with IL-12 monotherapy.

In still further embodiments, the present disclosure contemplates methods of treating or preventing a cancer-related disease, disorder or condition in a subject, comprising administering to the subject: a) a therapeutically effective amount of an IL-12 agent; and b) a therapeutically effective amount of a PEG-IL-10, wherein the amount is sufficient to i) maintain a mean IL-10 serum trough concentration over a period of time, wherein the mean IL-10 serum trough concentration is at least 1.0 ng/mL, and wherein the mean IL-10 serum trough concentration is maintained for at least 90% of the period of time; and ii) reduce the IL-12—associated toxicity to a level less than that observed with IL-12 monotherapy.

The desired IL-10 serum trough concentration may depend on a number of factors, including the nature of the disease, disorder or condition (e.g., localized tumor or metastatic disease), the extent to which the subject is suffering from the malady (e.g., early versus late stage disease), whether combination therapy is being administered, and patient-specific parameters (e.g., hepatic and renal function). By way of example, co-administration of PEG-IL-10 and a chemotherapeutic agent may only require a serum trough in the ˜1-2 ng/mL range in order to observe clinical benefit, while metastatic cancer may require 6-10 ng/mL or more to achieve comparable clinical benefit (see, e.g., WO 2014/172392). As the skilled artisan will appreciate, the desired serum trough levels are context-dependent (e.g., characteristics of a specific cancer) and patient-specific

Thus, in particular embodiments of the present disclosure, the mean IL-10 serum trough concentration is at least 1.0 ng/mL, at least 1.5 ng/mL, at least 2.0 ng/mL, at least 2.5 ng/mL, at least 3.0 ng/mL, at least 3.5 ng/mL, at least 4.0 ng/mL, at least 4.5 ng/mL, at least 5.0 ng/mL, and least 5.5 ng/mL, at least 6.0 ng/mL, at least 6.5 ng/Ml, at least 7.0 ng/mL, at least 7.5 ng/mL, at least 8.0 ng/mL, and least 9.0 ng/mL, at least 10.0 ng/mL, at least 11.0 ng/mL, at least 12.0 ng/mL, at least 13.0 ng/mL, at least 14.0 ng/mL, at least 15.0 ng/mL, at least 16.0 ng/mL, at least 17.0 ng/mL, at least 18.0 ng/mL, at least 19.0 ng/mL, at least 20.0 ng/mL, at least 21.0 ng/mL, at least 22.0 ng/mL, or greater than 22.0 ng/mL.

In further embodiments, the period of time is at least 12 hours, at least 24 hours, at least 48 hours, at least 72 hours, at least 1 week, at least 2 weeks, at least 3 weeks, at least 1 month, at least 6 weeks, at least 2 months, at least 3 months, or greater than 3 months.

In particular embodiments of the present disclosure, the mean IL-10 serum trough concentration is maintained for at least 85% of the period of time, at least 90%, at least 92.5%, at least 95%, at least 98%, at least 99% or 100% of the period of time.

It is envisaged that a dosing regimen sufficient to maintain a particular steady state serum trough concentration (e.g., 2.0 ng/mL) may result in an initial serum trough concentration that is higher than the desired steady state serum trough concentration. Because of the pharmacodynamic and pharmacokinetic characteristics of IL-10 in a mammalian subject, an initial trough concentration (achieved, for example, through the administration of one or more loading doses followed by a series of maintenance doses) gradually but continually decreases over a period of time even when the dosing parameters (amount and frequency) are kept constant. After that period of time, the gradual but continual decrease ends and a steady state serum trough concentration is maintained.

By way of example, parenteral administration (e.g., SC and IV) of ˜0.1 mg/kg/day of an IL-10 agent (e.g., mIL-10) to a mouse (e.g., a C57BL/6 mouse) is required to maintain a steady state serum trough concentration of, for example, 2.0 ng/mL. However, that steady state serum trough concentration may not be achieved until approximately 30 days after initiation of dosing at 0.1 mg/kg/day (and also after any loading dose(s)). Rather, after an initial serum trough concentration has been achieved (e.g., 2.5 ng/mL), that concentration gradually but continually decreases over the course of, for example, the approximately 30-day period, after which time the desired steady state serum trough concentration (e.g., 2.0 ng/mL) is maintained. One of skill in the art will be able to determine the dose needed to maintain the desired steady state trough concentration using, for example, ADME and patient-specific parameters.

A PEG-IL-10 of the present disclosure may comprise at least one PEG molecule covalently attached to at least one amino acid residue of at least one subunit of IL-10 or comprise a mixture (e.g., 1:1) of mono-pegylated and di-pegylated IL-10 in other embodiments. The PEG component of a PEG-IL-10 may have a molecular mass greater than about 5 kDa, greater than about 10 kDa, greater than about 15 kDa, greater than about 20 kDa, greater than about 30 kDa, greater than about 40 kDa, or greater than about 50 kDa. In some embodiments, the molecular mass is from about 5 kDa to about 10 kDa, from about 5 kDa to about 15 kDa, from about 5 kDa to about 20 kDa, from about 10 kDa to about 15 kDa, from about 10 kDa to about 20 kDa, from about 10 kDa to about 25 kDa or from about 10 kDa to about 30 kDa.

As indicated herein, the PEG-IL-10 is mature human PEG-IL-10 in some embodiments, while in other embodiments it is a variant of mature human PEG-IL-10 that exhibits activity comparable to the activity of mature human PEG-IL-10.

The present disclosure contemplates embodiments wherein the amount of the PEG-IL-10 component of the combination therapy that is administered to the subject to treat or prevent a cancer-related disease, disorder or condition is from 10.0 μg/kg/day to 20.0 μg/kg/day. In some embodiments, the amount of the PEG-IL-10 administered is from 12.0 μg/kg/day to 18.0 μg/kg/day. In some embodiments, the amount is less than 10.0 μg/kg/day, while in other embodiments it is greater than 20.0 μg/kg/day.

According to the present disclosure, a PEG-IL-10 may be administered in combination with an IL-12 agent for the treatment of a cancer-related disease, disorder or condition in the subject. A detailed description of the foregoing diseases, disorders and conditions is set forth elsewhere herein. In some embodiments, the cancer is a solid tumor, such as a tumor associated with breast cancer, prostate cancer, lung cancer, liver cancer, pancreatic cancer, brain cancer, stomach cancer, ovarian cancer, kidney cancer, testicular cancer, and melanoma. In particular embodiments, the cancer is a hematological disorder, including lymphomas such as a B-cell lymphoma, or a leukemia.

In particular embodiments of the present disclosure, the cancer-related disease, disorder or condition is an immune-insensitive tumor. Tumors that are insensitive to therapeutic immune manipulation may be described as exhibiting the following two characteristics: 1) active suppression of the immune system, and 2) an inflammatory response accompanied by the concomitant activation of immune-suppressive mechanisms resulting from treatment thereof (Galon et al. (Jul. 25 2013) Immunity 39:11-26 (PubMed PMID: 23890060)). Examples of immune-insensitive tumors include, but are not limited to, colon, gastroesophageal, pancreatic and breast cancer.

In certain embodiments of the present disclosure, the therapeutic effects of the PEG-IL-10 and the IL-12 agent are additive, while in other embodiments they are synergistic.

A PEG-IL-10 and an IL-12 agent may be administered by any effective route. In some embodiments, they are administered by parenteral injection, including subcutaneous injection. In particular embodiments, a PEG-IL-10 is administered separately from the IL-12 agent, and in other embodiments a PEG-IL-10 and an IL-12 agent are administered together. As indicated herein, for purposes of the present disclosure PEG-IL-10 and an IL-12 agent are deemed to be co-administered when administered separately or together, or in one or more means of delivery (e.g., a vial, IV bag or syringe).

As noted above, the various types of IL-12 agents for use in the combination therapies of the present disclosure include human and non-human IL-12—related polypeptides, including homologs, variants (including muteins), and fragments thereof. Also contemplated herein are functionally active components of the IL-12 complex, as well as the active heterodimer (p′70). In some embodiments, the IL-12 peptides have at least 85%, at least 87%, at least 90%, at least 91%, at least 92%, at least 93%, at least 94%, at least 95%, at least 96%, at least 97%, at least 98%, or at least 99% of the 306 amino acid residue human IL-12A polypeptide and/or of the 197 amino acid residue human IL-12B polypeptide. In other embodiments, the IL-12 peptides have at least 80%, at least 85%, at least 90%, at least 91%, at least 92%, at least 93%, at least 94%, at least 95%, at least 96%, at least 97%, at least 98%, or at least 99% sequence identity to the 306 amino acid residue human IL-12A polypeptide and/or of the 197 amino acid residue human IL-12B polypeptide.

As indicated herein, the IL-12 agent is mature human IL-12 in some embodiments, while in other embodiments the IL-12 agent is a variant of mature human IL-12 that exhibits activity comparable to the activity of mature human IL-12.

Also provided herein are embodiments wherein the amount of the IL-12 agent of the combination therapy that is administered to the subject to treat or prevent a cancer-related disease, disorder or condition is from 0.01 μg/kg/day to 10.0 μg/kg/day, from 0.1 μg/kg/day to 10.0 μg/kg/day, or from 1.0 μg/kg/day to 10.0 μg/kg/day. In some embodiments, the amount is less than 0.01 μg/kg/day, while in other embodiments it is greater than 10.0 μg/kg/day.

In particular embodiments, the present disclosure contemplates dosing an IL-12 agent such that the serum concentration achieves a peak and is then cleared such that it is essentially unmeasurable before it is administered again. In some embodiments, IL-12 treatment is initiated with a loading dose, followed by a series of maintenance doses, which may be at defined intervals. In order to avoid potential toxicities, dosing should be adjusted such that the IL-12 level does not exceed its maximally tolerated level. As with administration of a PEG-IL-10, the dose of an IL-12 agent may depend on a number of factors, including the nature of the disease, disorder or condition (e.g., localized tumor or metastatic disease), the extent to which the subject is suffering from the malady (e.g., early versus late stage disease), whether combination therapy is being administered, and patient-specific parameters (e.g., hepatic and renal function).

The present disclosure includes pharmaceutical compositions comprising a PEG-IL-10 and an IL-12 agent as described herein, and a pharmaceutically acceptable diluent, carrier or excipient. In some embodiments, the PEG-IL-10 and the IL-12 agent are present in separate pharmaceutical compositions, each comprising a pharmaceutically acceptable diluent, carrier or excipient. In some embodiments, the excipient is an isotonic injection solution. The pharmaceutical compositions may be suitable for administration to a subject (e.g., a human), and may comprise one or more additional prophylactic or therapeutic agents. In certain embodiments, the pharmaceutical compositions are contained in one or more sterile containers (e.g., a single- or multi-use vial or a syringe). A kit may contain the sterile container(s), and the kit may also contain one or more additional sterile containers comprising at least one additional prophylactic or therapeutic agent or any other agent that may be used in pharmacological therapy. One or more additional prophylactic or therapeutic agents may be administered prior to, simultaneously with, or subsequent to the PEG-IL-10 and IL-12 agent.

Additional prophylactic or therapeutic agents (also referred to herein as supplementary agents and the like) that may be used with the methods of treating and/or preventing a cancer-related disease, disorder or condition include any agent that may provide some therapeutic benefit. By way of example, but not limitation, a prophylactic or therapeutic agent may be a chemotherapeutic agent, an immune- or inflammation-related agent, a metabolic agent, an antiviral agent or an anti-thrombotic agent. The methods of the present disclosure may also be used in combination with non-pharmacological agents (e.g., radiology).

In particular embodiments, the additional prophylactic or therapeutic agent is a chemotherapeutic agent, examples of which are set forth herein. In some embodiments, the chemotherapeutic agent is a platinum-based antineoplastic, also referred to as a platinum coordination complex. These platinum-based antineoplastic agents crosslink DNA, thereby inhibiting DNA repair and/or DNA synthesis in cancer cells. Examples of such agents include cisplatin, carboplatin, oxaliplatin, satraplatin, picoplatin, nedaplatin and triplatin.

Methods and models for optimizing dosing regimens for the PEG-IL-10 and IL-12 agents described herein are also contemplated by embodiments of the present disclosure. In other embodiments, the present disclosure contemplates methods for the identification of specific patient populations that are optimally suited for the combination therapies described herein. In some embodiments, the existence and/or extent of certain biomarkers can find utility in such methods.

Other aspects and embodiments will be apparent to the skilled artisan after reviewing the present disclosure.

### BRIEF DESCRIPTION OF THE DRAWINGS

FIG. 1 depicts the amino acid sequences of Human IL-12, Chain A (SEQ ID NO:1); and Human IL-12, Chain B (SEQ ID NO:2).

FIG. 2 depicts the effect of PEG-rMuIL-10 (1 mg/kg) and/or rMuIL-12 (0.05, 0.1, or 0.5 mg/kg) administered SC daily for 21 days as monotherapy or as combination therapy in 4T1 tumor-bearing mice. Tumor weights were assessed after study completion.

FIGS. 3A and 3B depict the effect of PEG-rMuIL-10 (1 mg/kg) and/or rMuIL-12 (0.05, 0.1, or 0.5 mg/kg) administered SC daily as monotherapy or as combination therapy to 4T1 tumor-bearing mice on serum IFNγ (FIG. 3A) and on serum TNFα (FIG. 3B). Serum IFNγ and TNFα levels were assessed after 9 days of dosing, 4 hrs after dose administration.

### DETAILED DESCRIPTION

Before the present disclosure is further described, it is to be understood that the disclosure is not limited to the particular embodiments set forth herein, and it is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention. Unless defined otherwise, all technical and scientific terms used herein have the same meaning as commonly understood by one of ordinary skill in the art to which this invention belongs.

It must be noted that as used herein and in the appended claims, the singular forms “a,” “an,” and “the” include plural referents unless the context clearly dictates otherwise. It is further noted that the claims may be drafted to exclude any optional element. As such, this statement is intended to serve as antecedent basis for use of such exclusive terminology such as “solely,” “only” and the like in connection with the recitation of claim elements, or use of a “negative” limitation.

The publications discussed herein are provided solely for their disclosure prior to the filing date of the present application. Further, the dates of publication provided may be different from the actual publication dates, which may need to be independently confirmed.

### Overview

As described herein, the inventors of the present disclosure have discovered that co-administration of PEG-IL-10 and IL-12 under certain conditions and parameters can temper IL-12's untoward adverse effects while still retaining its potent anti-tumor activity. In view of that finding, the present disclosure contemplates methods of using a PEG-IL-10 (e.g., rHuPEG-IL-10), and compositions thereof, in combination with an IL-12 agent (e.g., rHuIL-12), and compositions thereof, for the treatment and/or prevention of cancer-related diseases, disorders and conditions, and/or the symptoms thereof. The methods comprise particular dosing regimens and provide the opportunity for additive or synergistic effects in the treatment and/or prevention of the disorders described herein.

It should be noted that any reference to “human” in connection with the polypeptides and nucleic acid molecules of the present disclosure is not meant to be limiting with respect to the manner in which the polypeptide or nucleic acid is obtained or the source, but rather is only with reference to the sequence as it can correspond to a sequence of a naturally occurring human polypeptide or nucleic acid molecule. In addition to the human polypeptides and the nucleic acid molecules which encode them, the present disclosure contemplates IL-10—and IL-12—related polypeptides and corresponding nucleic acid molecules from other species.

### Definitions

Unless otherwise indicated, the following terms are intended to have the meaning set forth below. Other terms are defined elsewhere throughout the specification.

The terms “patient” or “subject” are used interchangeably to refer to a human or a non-human animal (e.g., a mammal).

The terms “administration”, “administer” and the like, as they apply to, for example, a subject, cell, tissue, organ, or biological fluid, refer to contact of, for example, IL-10 or PEG-IL-10), a nucleic acid (e.g., a nucleic acid encoding native human IL-10); a pharmaceutical composition comprising the foregoing, or a diagnostic agent to the subject, cell, tissue, organ, or biological fluid. In the context of a cell, administration includes contact (e.g., in vitro or ex vivo) of a reagent to the cell, as well as contact of a reagent to a fluid, where the fluid is in contact with the cell.

The terms “treat”, “treating”, treatment” and the like refer to a course of action (such as administering IL-10 or a pharmaceutical composition comprising IL-10) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject. Thus, treatment includes inhibiting (e.g., arresting the development or further development of the disease, disorder or condition or clinical symptoms association therewith) an active disease. The terms may also be used in other contexts, such as situations where IL-10 or PEG-IL-10 contacts an IL-10 receptor in, for example, the fluid phase or colloidal phase.

The term “in need of treatment” as used herein refers to a judgment made by a physician or other caregiver that a subject requires or will benefit from treatment. This judgment is made based on a variety of factors that are in the realm of the physician's or caregiver's expertise.

The terms “prevent”, “preventing”, “prevention” and the like refer to a course of action (such as administering IL-10 or a pharmaceutical composition comprising IL-10) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The term “in need of prevention” as used herein refers to a judgment made by a physician or other caregiver that a subject requires or will benefit from preventative care. This judgment is made based on a variety of factors that are in the realm of a physician's or caregiver's expertise.

The phrase “therapeutically effective amount” refers to the administration of an agent to a subject, either alone or as part of a pharmaceutical composition and either in a single dose or as part of a series of doses, in an amount capable of having any detectable, positive effect on any symptom, aspect, or characteristic of a disease, disorder or condition when administered to the subject. The therapeutically effective amount can be ascertained by measuring relevant physiological effects, and it can be adjusted in connection with the dosing regimen and diagnostic analysis of the subject's condition, and the like. By way of example, measurement of the amount of inflammatory cytokines produced following administration can be indicative of whether a therapeutically effective amount has been used.

The phrase “in a sufficient amount to effect a change” means that there is a detectable difference between a level of an indicator measured before (e.g., a baseline level) and after administration of a particular therapy. Indicators include any objective parameter (e.g., serum concentration of IL-10) or subjective parameter (e.g., a subject's feeling of well-being).

The term “small molecules” refers to chemical compounds having a molecular weight that is less than about 10 kDa, less than about 2 kDa, or less than about 1 kDa. Small molecules include, but are not limited to, inorganic molecules, organic molecules, organic molecules containing an inorganic component, molecules comprising a radioactive atom, and synthetic molecules. Therapeutically, a small molecule can be more permeable to cells, less susceptible to degradation, and less likely to elicit an immune response than large molecules.

The term “ligand” refers to, for example, peptide, polypeptide, membrane-associated or membrane-bound molecule, or complex thereof, that can act as an agonist or antagonist of a receptor. “Ligand” encompasses natural and synthetic ligands, e.g., cytokines, cytokine variants, analogs, muteins, and binding compositions derived from antibodies. “Ligand” also encompasses small molecules, e.g., peptide mimetics of cytokines and peptide mimetics of antibodies. The term also encompasses an agent that is neither an agonist nor antagonist, but that can bind to a receptor without significantly influencing its biological properties, e.g., signaling or adhesion. Moreover, the term includes a membrane-bound ligand that has been changed, e.g., by chemical or recombinant methods, to a soluble version of the membrane-bound ligand. A ligand or receptor can be entirely intracellular, that is, it can reside in the cytosol, nucleus, or some other intracellular compartment. The complex of a ligand and receptor is termed a “ligand-receptor complex.”

The terms “inhibitors” and “antagonists”, or “activators” and “agonists”, refer to inhibitory or activating molecules, respectively, for example, for the activation of, e.g., a ligand, receptor, cofactor, gene, cell, tissue, or organ. Inhibitors are molecules that decrease, block, prevent, delay activation, inactivate, desensitize, or down-regulate, e.g., a gene, protein, ligand, receptor, or cell. Activators are molecules that increase, activate, facilitate, enhance activation, sensitize, or up-regulate, e.g., a gene, protein, ligand, receptor, or cell. An inhibitor can also be defined as a molecule that reduces, blocks, or inactivates a constitutive activity. An “agonist” is a molecule that interacts with a target to cause or promote an increase in the activation of the target. An “antagonist” is a molecule that opposes the action(s) of an agonist. An antagonist prevents, reduces, inhibits, or neutralizes the activity of an agonist, and an antagonist can also prevent, inhibit, or reduce constitutive activity of a target, e.g., a target receptor, even where there is no identified agonist.

The terms “modulate”, “modulation” and the like refer to the ability of a molecule (e.g., an activator or an inhibitor) to increase or decrease the function or activity of a PEG-IL-10 (or the nucleic acid molecules encoding them), either directly or indirectly; or to enhance the ability of a molecule to produce an effect comparable to that of a PEG-IL-10. The term “modulator” is meant to refer broadly to molecules that can effect the activities described above. By way of example, a modulator of, e.g., a gene, a receptor, a ligand, or a cell, is a molecule that alters an activity of the gene, receptor, ligand, or cell, where activity can be activated, inhibited, or altered in its regulatory properties. A modulator can act alone, or it can use a cofactor, e.g., a protein, metal ion, or small molecule. The term “modulator” includes agents that operate through the same mechanism of action as IL-10 (i.e., agents that modulate the same signaling pathway as IL-10 in a manner analogous thereto) and are capable of eliciting a biological response comparable to (or greater than) that of IL-10.

Examples of modulators include small molecule compounds and other bioorganic molecules. Numerous libraries of small molecule compounds (e.g., combinatorial libraries) are commercially available and can serve as a starting point for identifying a modulator. The skilled artisan is able to develop one or more assays (e.g., biochemical or cell-based assays) in which such compound libraries can be screened in order to identify one or more compounds having the desired properties; thereafter, the skilled medicinal chemist is able to optimize such one or more compounds by, for example, synthesizing and evaluating analogs and derivatives thereof. Synthetic and/or molecular modeling studies can also be utilized in the identification of an Activator.

The “activity” of a molecule can describe or refer to the binding of the molecule to a ligand or to a receptor; to catalytic activity; to the ability to stimulate gene expression or cell signaling, differentiation, or maturation; to antigenic activity; to the modulation of activities of other molecules; and the like. The term can also refer to activity in modulating or maintaining cell-to-cell interactions (e.g., adhesion), or activity in maintaining a structure of a cell (e.g., a cell membrane). “Activity” can also mean specific activity, e.g., [catalytic activity]/[mg protein], or [immunological activity]/[mg protein], concentration in a biological compartment, or the like. The term “proliferative activity” encompasses an activity that promotes, that is necessary for, or that is specifically associated with, for example, normal cell division, as well as cancer, tumors, dysplasia, cell transformation, metastasis, and angiogenesis.

As used herein, “comparable”, “comparable activity”, “activity comparable to”, “comparable effect”, “effect comparable to”, and the like are relative terms that can be viewed quantitatively and/or qualitatively. The meaning of the terms is frequently dependent on the context in which they are used. By way of example, two agents that both activate a receptor can be viewed as having a comparable effect from a qualitative perspective, but the two agents can be viewed as lacking a comparable effect from a quantitative perspective if one agent is only able to achieve 20% of the activity of the other agent as determined in an art-accepted assay (e.g., a dose-response assay) or in an art-accepted animal model. When comparing one result to another result (e.g., one result to a reference standard), “comparable” frequently means that one result deviates from a reference standard by less than 35%, by less than 30%, by less than 25%, by less than 20%, by less than 15%, by less than 10%, by less than 7%, by less than 5%, by less than 4%, by less than 3%, by less than 2%, or by less than 1%. In particular embodiments, one result is comparable to a reference standard if it deviates by less than 15%, by less than 10%, or by less than 5% from the reference standard. By way of example, but not limitation, the activity or effect can refer to efficacy, stability, solubility, or immunogenicity.

The term “response,” for example, of a cell, tissue, organ, or organism, encompasses a change in biochemical or physiological behavior, e.g., concentration, density, adhesion, or migration within a biological compartment, rate of gene expression, or state of differentiation, where the change is correlated with activation, stimulation, or treatment, or with internal mechanisms such as genetic programming. In certain contexts, the terms “activation”, “stimulation”, and the like refer to cell activation as regulated by internal mechanisms, as well as by external or environmental factors; whereas the terms “inhibition”, “down-regulation” and the like refer to the opposite effects.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence; fusion proteins with heterologous and homologous leader sequences; fusion proteins with or without N-terminus methionine residues; fusion proteins with immunologically tagged proteins; and the like.

It will be appreciated that throughout this disclosure reference is made to amino acids according to the single letter or three letter codes. For the reader's convenience, the single and three letter amino acid codes are provided below:

| | | | | | |
|---|---|---|---|---|---|
| G | Glycine | Gly | P | Proline | Pro |
| A | Alanine | Ala | V | Valine | Val |
| L | Leucine | Leu | I | Isoleucine | Ile |
| M | Methionine | Met | C | Cysteine | Cys |
| F | Phenylalanine | Phe | Y | Tyrosine | Tyr |
| W | Tryptophan | Trp | H | Histidine | His |
| K | Lysine | Lys | R | Arginine | Arg |
| Q | Glutamine | Gln | N | Asparagine | Asn |
| E | Glutamic Acid | Glu | D | Aspartic Acid | Asp |
| S | Serine | Ser | T | Threonine | Thr |

As used herein, the term “variant” encompasses naturally-occurring variants and non-naturally-occurring variants. Naturally-occurring variants include homologs (polypeptides and nucleic acids that differ in amino acid or nucleotide sequence, respectively, from one species to another), and allelic variants (polypeptides and nucleic acids that differ in amino acid or nucleotide sequence, respectively, from one individual to another within a species). Non-naturally-occurring variants include polypeptides and nucleic acids that comprise a change in amino acid or nucleotide sequence, respectively, where the change in sequence is artificially introduced (e.g., muteins); for example, the change is generated in the laboratory by human intervention (“hand of man”). Thus, herein a “mutein” refers broadly to mutated recombinant proteins that usually carry single or multiple amino acid substitutions and are frequently derived from cloned genes that have been subjected to site-directed or random mutagenesis, or from completely synthetic genes.

The terms “DNA”, “nucleic acid”, “nucleic acid molecule”, “polynucleotide” and the like are used interchangeably herein to refer to a polymeric form of nucleotides of any length, either deoxyribonucleotides or ribonucleotides, or analogs thereof. Non-limiting examples of polynucleotides include linear and circular nucleic acids, messenger RNA (mRNA), complementary DNA (cDNA), recombinant polynucleotides, vectors, probes, primers and the like.

As used herein in the context of the structure of a polypeptide, “N-terminus” (or “amino terminus”) and “C-terminus” (or “carboxyl terminus”) refer to the extreme amino and carboxyl ends of the polypeptide, respectively, while the terms “N-terminal” and “C-terminal” refer to relative positions in the amino acid sequence of the polypeptide toward the N-terminus and the C-terminus, respectively, and can include the residues at the N-terminus and C-terminus, respectively. “Immediately N-terminal” or “immediately C-terminal” refers to a position of a first amino acid residue relative to a second amino acid residue where the first and second amino acid residues are covalently bound to provide a contiguous amino acid sequence.

“Derived from”, in the context of an amino acid sequence or polynucleotide sequence (e.g., an amino acid sequence “derived from” an IL-10 polypeptide), is meant to indicate that the polypeptide or nucleic acid has a sequence that is based on that of a reference polypeptide or nucleic acid (e.g., a naturally occurring IL-10 polypeptide or an IL-10-encoding nucleic acid), and is not meant to be limiting as to the source or method in which the protein or nucleic acid is made. By way of example, the term “derived from” includes homologs or variants of reference amino acid or DNA sequences.

In the context of a polypeptide, the term “isolated” refers to a polypeptide of interest that, if naturally occurring, is in an environment different from that in which it can naturally occur. “Isolated” is meant to include polypeptides that are within samples that are substantially enriched for the polypeptide of interest and/or in which the polypeptide of interest is partially or substantially purified. Where the polypeptide is not naturally occurring, “isolated” indicates that the polypeptide has been separated from an environment in which it was made by either synthetic or recombinant means.

“Enriched” means that a sample is non-naturally manipulated (e.g., by a scientist) so that a polypeptide of interest is present in a) a greater concentration (e.g., at least 3-fold greater, at least 4-fold greater, at least 8-fold greater, at least 64-fold greater, or more) than the concentration of the polypeptide in the starting sample, such as a biological sample (e.g., a sample in which the polypeptide naturally occurs or in which it is present after administration), or b) a concentration greater than the environment in which the polypeptide was made (e.g., as in a bacterial cell).

“Substantially pure” indicates that a component (e.g., a polypeptide) makes up greater than about 50% of the total content of the composition, and typically greater than about 60% of the total polypeptide content. More typically, “substantially pure” refers to compositions in which at least 75%, at least 85%, at least 90% or more of the total composition is the component of interest. In some cases, the polypeptide will make up greater than about 90%, or greater than about 95% of the total content of the composition.

The terms “specifically binds” or “selectively binds”, when referring to a ligand/receptor, antibody/antigen, or other binding pair, indicates a binding reaction which is determinative of the presence of the protein in a heterogeneous population of proteins and other biologics. Thus, under designated conditions, a specified ligand binds to a particular receptor and does not bind in a significant amount to other proteins present in the sample. The antibody, or binding composition derived from the antigen-binding site of an antibody, of the contemplated method binds to its antigen, or a variant or mutein thereof, with an affinity that is at least two-fold greater, at least ten times greater, at least 20-times greater, or at least 100-times greater than the affinity with any other antibody, or binding composition derived therefrom. In a particular embodiment, the antibody will have an affinity that is greater than about 10⁹ liters/mol, as determined by, e.g., Scatchard analysis (Munsen, et al. 1980 Analyt. Biochem. 107:220-239).

### IL-10 and PEG-IL-10

The anti-inflammatory cytokine IL-10, also known as human cytokine synthesis inhibitory factor (CSIF), is classified as a type(class)-2 cytokine, a set of cytokines that includes IL-19, IL-20, IL-22, IL-24 (Mda-7), and IL-26, interferons (IFN-α, −β, −γ, −δ, −ε, −κ and −κ) and interferon-like molecules (limitin, IL-28A, IL-28B, and IL-29).

IL-10 is a cytokine with pleiotropic effects in immunoregulation and inflammation. It is produced by mast cells, counteracting the inflammatory effect that these cells have at the site of an allergic reaction. While it is capable of inhibiting the synthesis of pro-inflammatory cytokines such as IFN-γ, IL-2, IL-3, TNFα and GM-CSF, IL-10 is also stimulatory towards certain T cells and mast cells and stimulates B-cell maturation, proliferation and antibody production. IL-10 can block NF-κB activity and is involved in the regulation of the JAK-STAT signaling pathway. It also induces the cytotoxic activity of CD8+ T-cells and the antibody production of B-cells, and it suppresses macrophage activity and tumor-promoting inflammation. The regulation of CD8+ T-cells is dose-dependent, wherein higher doses induce stronger cytotoxic responses.

As indicated elsewhere herein, IL-10 is deemed to be an anti-inflammatory and immuno-suppressive cytokine that inhibits the secretion of IFN-γ, IL-12 (D'Andrea, A., et al. (1993) J Exp Med 178(3):1041-48), and TNFα (Armstrong, L., et al. (1996) Thorax 51(2):143-49). IL-10 also inhibits antigen presentation and subsequent activation of CD4+ T cells (de Waal Malefyt, R., et al. (1991) J Exp Med 174(5):1209-20; de Waal Malefyt, R., et al. (1991) J Exp Med 174(4):915-24) and is thus widely considered to be a potent immune suppressive cytokine.

Human IL-10 is a homodimer with a molecular mass of 37 kDa, wherein each 18.5 kDa monomer comprises 178 amino acids, the first 18 of which comprise a signal peptide, and two cysteine residues that form two intramolecular disulfide bonds. The IL-10 dimer becomes biologically inactive upon disruption of the non-covalent interactions between the two monomer subunits.

As alluded to above, the terms “IL-10”, “IL-10 polypeptide(s), “IL-10 molecule(s)”, “IL-10 agent(s)” and the like are intended to be broadly construed and include, for example, human and non-human IL-10—related polypeptides, including homologs, variants (including muteins), and fragments thereof, as well as IL-10 polypeptides having, for example, a leader sequence (e.g., the signal peptide), and modified versions of the foregoing. The present disclosure contemplates pegylated forms of human IL-10 (NP_000563) and murine IL-10 (NP_034678), which exhibit 80% homology, and use thereof. In addition, the scope of the present disclosure includes pegylated IL-10 orthologs, and modified forms thereof, from other mammalian species, including rat (accession NP_036986.2; GI 148747382); cow (accession NP_776513.1; GI 41386772); sheep (accession NP_001009327.1; GI 57164347); dog (accession ABY86619.1; GI 166244598); and rabbit (accession AAC23839.1; GI 3242896).

The IL-10 receptor, a type II cytokine receptor, consists of alpha and beta subunits, which are also referred to as R1 and R2, respectively. Receptor activation requires binding to both alpha and beta. One homodimer of an IL-10 polypeptide binds to alpha and the other homodimer of the same IL-10 polypeptide binds to beta.

As used herein, the terms “pegylated IL-10”, “PEG-IL-10” and the like refer to an IL-10 molecule having one or more polyethylene glycol molecules covalently attached to at least one amino acid residue of the IL-10 protein, generally via a linker, such that the attachment is stable. The terms “monopegylated IL-10” and “mono-PEG-IL-10” indicate that one polyethylene glycol molecule is covalently attached to a single amino acid residue on one subunit of the IL-10 dimer, generally via a linker. As used herein, the terms “dipegylated IL-10” and “di-PEG-IL-10” indicate that at least one polyethylene glycol molecule is attached to a single residue on each subunit of the IL-10 dimer, generally via a linker.

In certain embodiments, the PEG-IL-10 used in the present disclosure is a mono-PEG-IL-10 in which one to nine PEG molecules are covalently attached via a linker to the alpha amino group of the amino acid residue at the N-terminus of one subunit of the IL-10 dimer. Monopegylation on one IL-10 subunit generally results in a non-homogeneous mixture of non-pegylated, monopegylated and dipegylated IL-10 due to subunit shuffling. Moreover, allowing a pegylation reaction to proceed to completion will generally result in non-specific and multi-pegylated IL-10, thus reducing its bioactivity. Thus, particular embodiments of the present disclosure comprise the administration of a mixture of mono- and di-pegylated IL-10 produced by the methods described herein.

In particular embodiments, the average molecular weight of the PEG moiety is between about 5 kDa and about 50 kDa. Although the method or site of PEG attachment to IL-10 is not critical, in certain embodiments the pegylation does not alter, or only minimally alters, the activity of the IL-10 peptide. In certain embodiments, the increase in half-life is greater than any decrease in biological activity. The biological activity of PEG-IL-10 is typically measured by assessing the levels of inflammatory cytokines (e.g., TNFα or IFNγ) in the serum of subjects challenged with a bacterial antigen (lipopolysaccharide (LPS)) and treated with PEG-IL-10, as described in U.S. Pat. No. 7,052,686.

IL-10 variants can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are usually predetermined variants not found in nature, although some can be post-translational variants, e.g., glycosylated variants. The present disclosure contemplates the use of any pegylated variant of IL-10 provided it retains a suitable level of IL-10 activity.

The phrase “conservative amino acid substitution” refers to substitutions that preserve the activity of the protein by replacing an amino acid(s) in the protein with an amino acid with a side chain of similar acidity, basicity, charge, polarity, or size of the side chain. Conservative amino acid substitutions generally entail substitution of amino acid residues within the following groups: 1) L, I, M, V, F; 2) R, K; 3) F, Y, H, W, R; 4) G, A, T, S; 5) Q, N; and 6) D, E. Guidance for substitutions, insertions, or deletions can be based on alignments of amino acid sequences of different variant proteins or proteins from different species. Thus, in addition to any naturally-occurring IL-10 polypeptide, the present disclosure contemplates having 1, 2, 3, 4, 5, 6, 7, 8, 9, or 10 usually no more than 20, 10, or 5 amino acid substitutions, where the substitution is usually a conservative amino acid substitution.

The present disclosure also contemplates pegylated forms of active fragments (e.g., subsequences) of mature IL-10 containing contiguous amino acid residues derived from the mature IL-10. The length of contiguous amino acid residues of a peptide or a polypeptide subsequence varies depending on the specific naturally-occurring amino acid sequence from which the subsequence is derived. In general, peptides and polypeptides can be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length peptide or polypeptide.

Additionally, IL-10 polypeptides can have a defined sequence identity compared to a reference sequence over a defined length of contiguous amino acids (e.g., a “comparison window”). Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Madison, Wis.), or by manual alignment and visual inspection (see, e.g., Current Protocols in Molecular Biology (Ausubel et al., eds. 1995 supplement)).

As an example, a suitable IL-10 polypeptide that can be pegylated can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 150 amino acids, from about 150 amino acids to about 155 amino acids, from about 155 amino acids up to the full-length IL-10 peptide or polypeptide.

As discussed further below, the IL-10 polypeptides can be isolated from a natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-10 polypeptides can also be synthetically produced (e.g., by cell-free chemical synthesis).

Nucleic acid molecules encoding an IL-10 molecule are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-10 polypeptide due to degeneracy of the genetic code.

### IL-12

Interleukin-12 (IL-12) is a pleiotropic cytokine naturally produced by macrophages, B-lymphoblastoid cells, dendritic cells, and neutrophils in response to antigenic stimulation. It was first described as a factor secreted from PMA-induced EBV-transformed B-cell lines. IL-12 is involved in the differentiation of naïve T cells into Th1 cells, can stimulate the growth and function of T cells, and mediates enhancement of the cytotoxic activity of NK cells and CD8+ cytotoxic T lymphocytes. As such, IL-12 activates both innate (NK cells) and adaptive (cytotoxic T Lymphocytes). IL-12 also stimulates the production of IFNγ and TNFα from T cells and NK cells, and reduces IL-4—mediated suppression of IFNγ.

As indicated elsewhere herein, the terms “IL-12”, “IL-12 polypeptide(s),” “IL-12-agent(s)”, “IL-12 molecule(s)” and the like are intended to be construed broadly and include, for example, human and non-human IL-12—related polypeptides, including homologs, variants (including muteins), and fragments thereof, as well as IL-12 polypeptides having, for example, a leader sequence (e.g., a signal peptide).

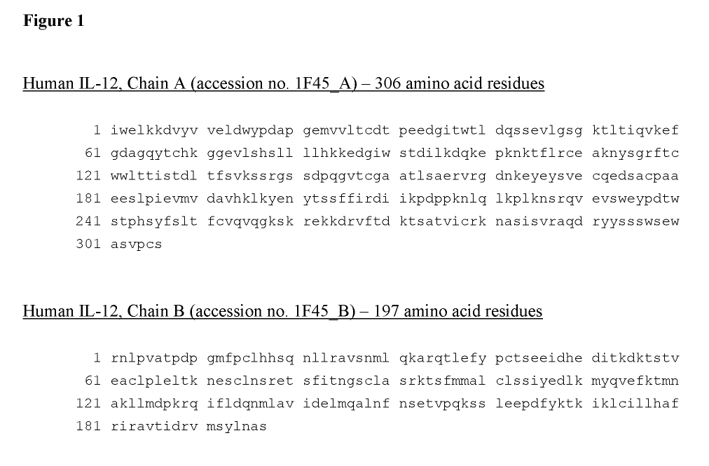

Structurally, IL-12 comprises a complex of four alpha helices. It is a heterodimeric cytokine encoded by two separate genes, IL-12, Chain A (p35) and IL-12, Chain B (p40). Human IL-12A is a 306 amino acid residue polypeptide (FIG. 1; accession no. 1F45 A), while human IL-12B is a 197 amino acid residue polypeptide (FIG. 1; accession no. 1F45_B). The active heterodimer (p′70) and a homodimer of p40 are formed following protein synthesis. IL-12 binds to the IL-12 receptor, a heterodimeric receptor formed by IL-12R-β1 and IL-12R-β2, which initiates a signaling cascade comprising several transcription factors involved in the JAK-STAT pathway.

The present disclosure contemplates active fragments (e.g., subsequences) of mature IL-12 containing contiguous amino acid residues derived from the mature IL-12. The length of contiguous amino acid residues of a peptide or a polypeptide subsequence varies depending on the specific naturally-occurring amino acid sequence from which the subsequence is derived. In general, peptides and polypeptides can be from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, from about 180 amino acids to about 190 amino acids, from about 190 amino acids to about 194 amino acids, from about 194 amino acids to about 196 amino acids, from about 196 amino acids to about 210 amino acids, from about 210 amino acids to about 230 amino acids, from about 230 amino acids to about 250 amino acids, from about 250 amino acids to about 270 amino acids, from about 270 amino acids to about 290 amino acids, from about 290 amino acids to about 295 amino acids, from about 295 amino acids to about 300 amino acids, from about 300 amino acids to about 304 amino acids, and from about 304 amino acids to about 306 amino acids.

Additionally, IL-12 polypeptides can have a defined sequence identity compared to a reference sequence over a defined length of contiguous amino acids (e.g., a “comparison window”). Methods of alignment of sequences for comparison are well-known in the art and are described above. As an example, a suitable IL-12 polypeptide can comprise an amino acid sequence having at least about 75%, at least about 80%, at least about 85%, at least about 90%, at least about 95%, at least about 98%, or at least about 99%, amino acid sequence identity to a contiguous stretch of from about 20 amino acids to about 40 amino acids, from about 40 amino acids to about 60 amino acids, from about 60 amino acids to about 80 amino acids, from about 80 amino acids to about 100 amino acids, from about 100 amino acids to about 120 amino acids, from about 120 amino acids to about 140 amino acids, from about 140 amino acids to about 160 amino acids, from about 160 amino acids to about 180 amino acids, from about 180 amino acids to about 190 amino acids, from about 190 amino acids to about 195 amino acids, from about 194 amino acids to about 196 amino acids, from about 196 amino acids to about 210 amino acids, from about 210 amino acids to about 230 amino acids, from about 230 amino acids to about 250 amino acids, from about 250 amino acids to about 270 amino acids, from about 270 amino acids to about 290 amino acids, from about 290 amino acids to about 295 amino acids, from about 295 amino acids to about 300 amino acids, from about 300 amino acids to about 304 amino acids, and from about 304 amino acids to about 306 amino acids.

As indicated elsewhere herein, the IL-12 polypeptides can be isolated from a natural source (e.g., an environment other than its naturally-occurring environment) and can also be recombinantly made (e.g., in a genetically modified host cell such as bacteria, yeast, Pichia, insect cells, and the like), where the genetically modified host cell is modified with a nucleic acid comprising a nucleotide sequence encoding the polypeptide. The IL-12 polypeptides can also be synthetically produced (e.g., by cell-free chemical synthesis).

Nucleic acid molecules encoding an IL-12 molecule are contemplated by the present disclosure, including their naturally-occurring and non-naturally occurring isoforms, allelic variants and splice variants. The present disclosure also encompasses nucleic acid sequences that vary in one or more bases from a naturally-occurring DNA sequence but still translate into an amino acid sequence that corresponds to an IL-12 polypeptide due to degeneracy of the genetic code.

IFNγ has been shown to coordinate natural mechanisms of anticancer defense (Jakobisiak, M. et al. (2013) Immunol Lett 90:103-22). By stimulating the production of IFNγ, IL-12 increases the production of the inducible protein-10 chemokine (IP-10 or CXCL10), which, in turn, mediates IL-12's anti-angiogenic effect. Because of its ability to induce immune responses and its anti-angiogenic activity, IL-12 has been evaluated as an oncology therapeutic. IL-12 may be useful in treating other disorders, including psoriasis and inflammatory bowel disease.

Of note, an anti-IL-12/23p40 neutralizing antibody (ustekinumab) has been evaluated in the clinic for the treatment of several immune-mediates disorders, including psoriasis, ankylosing spondylitis, rheumatoid arthritis, multiple sclerosis, atopic dermatitis, primary biliary cirrhosis, sarcoidosis and systemic lupus erythematosus. The most advances studies were directed to the treatment of psoriasis. (See, e.g., Teng, M. et al. (2015) Nature Medicine 21(7):719-29).

Due to its ability to interconnect the innate and adaptive immune arms and its potent stimulation of IFNγ production, IL-12 was initially thought to represent an ideal candidate for tumor immunotherapy. Indeed, results from early studies in animal models supported its potential use as a cancer therapeutic. However, clinical studies wherein IL-12 was administered systemically yielded a very narrow therapeutic index and resulted in substantial immune-related toxicity due to both significantly increased serum cytokines (primarily IFNγ and TNFα) and autoimmune hepatitis. Together, these cytokines lead to dose-limiting lethal toxicities and a maximally tolerated dose for IL-12 of 0.3 μg/kg subcutaneously daily (see Bajetta, E., et al., Clin Cancer Res, 1998. 4(1): p. 75-85; Motzer, R. J., et al., J Interferon Cytokine Res, 2001. 21(4): p. 257-63; Cebon, J., et al., Cancer Immun, 2003. 3: p. 7; Younes, A., et al., Clin Cancer Res, 2004. 10(16): p. 5432-8; and Ansell, S. M., et al., Blood, 2002. 99(1): p. 67-74). As a result, systemic administration of IL-12 in the oncology setting was largely deemed unviable. [See, e.g., Teng, M. et al. (2015) Nature Medicine 21(7):719-29].

In an effort to harness IL-12's anti-tumor effect and avoid its inherent shortcomings, alternative approaches to systemic administration have been explored. Autologous inactivated tumor cells expressing IL-12 and IL-10 were found to induce beneficial effects in mice with colon or mammary tumors and lung metastases (Lopez et al. (2005) J Immunol 175:5885-94). Despite this apparent positive effect, the report of this 2005 study did not result in a concerted effort to explore IL-10/IL-12 systemic combination therapy—likely due to the toxicity issues previously experienced with IL-12 in the clinical setting. As indicated above, IL-12—associated toxicities that have been observed include flu-like symptoms (e.g., headache, fever, chills, fatigue and arthromyalgia); hematologic toxicity, including neutropenia and thrombocytopenia; and hepatic toxicity, manifested by dose-dependent increases in transaminases, hyperbilirubinemia, and hypoalbuminemia. Other IL-12 associated adverse effects include inflammation of mucus membranes (e.g., oral mucositis, stomatitis and colitis), hypotension, renal impairment and gastrointestinal bleeding. These toxic effects have been associated with the secondary production of IFNγ and TNFα, as well as other cytokines (e.g., IP-10 and MIG). (See, e.g., Lasek, et al. (2014) Cancer Immunol Immunother 63:419-35; Xu, et al. Clinical and Developmental Immunology, volume 2010, Article ID 832454, 9 pp.); Cebon, J., et al., Cancer Immun, 2003. 3: p. 7).

Recent efforts to leverage IL-12's potential usefulness in oncology have taken different directions. For example, clinical studies have been initiated in which IL-12 is applied as an adjuvant in cancer vaccines, in gene therapy including loco regional injections of IL-12 plasmid, and in the form of tumor-targeting immunocytokines. Other strategies include co-administration with Treg cell-depleting antibodies (e.g., an anti-CD25 antibody), antibodies against immune suppressive signals (e.g., CTLA-4), and anticancer drugs. (See, e.g., Lasek, et al. (2014) Cancer Immunol Immunother 63:419-35; Xu, et al. Clinical and Developmental Immunology, volume 2010, Article ID 832454, 9 pp.)). These approaches make it further apparent that the oncology community has concluded that IL-10/IL-12 systemic combination therapy is intractable.

### Co-Administration of IL-10 and IL-12

The combination of PEG-IL-10 and IL-12 is thought to exhibit at least additive, and possibly synergistic, anti-tumor efficacy. However, the toxicity observed with IL-12 monotherapy has heretofore limited the exploration of such combination therapy in human subjects. In particular, IL-12 exhibits potent immunostimulatory biology which limits its maximally tolerated dose (which has been described as 0.5-1.25 μg/kg; see Cebon, J., et al., Cancer Immun, 2003. 3: p. 7) to an amount which is lower than its maximally efficacious dose. Although an understanding of the mechanism underlying this phenomenon is not required in order to practice the present disclosure, it is thought to be due to the activation by IL-12 of both antigen-non-specific naïve CD4+ and CD8+ T cells and antigen-specific CD4+ and CD8+ T cells, as well as NK cells. While IL-12 exhibits a broad spectrum immune stimulation that is both antigen-specific and antigen-non-specific, PEG-IL-10 exposure only activates the antigen-specific population of CD8+ T cells. As indicated below, when combined, PEG-IL-10 likely limits the non-antigen-specific immune stimulation of IL-12 and focuses IL-12's immunostimulatory effects into the antigen specific, adaptive CD8+ T cell arm of the immune system.

IL-12's immunostimulatory response comprises, in part, the induction of IFNγ and TNFα secretion from these cells, and this elevation of serum IFNγ, and to a lesser extent serum TNFα, is correlated with the onset of immune-related toxicities. Although PEG-IL-10 treatment also leads to the elevation of serum IFNγ levels, its MTD has not been established due to therapeutic benefit obtained at doses ranging from 5-40 μg/kg dosed subcutaneously daily.

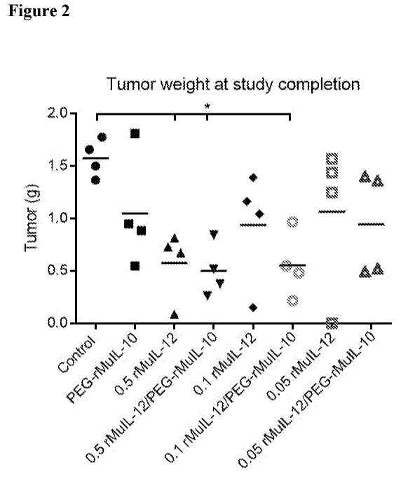

As described in detail in the Experimental section, the combinatorial effect of PEG-IL-10 and IL-12 on tumor size in a murine 4T1 tumor model was evaluated. As indicated in FIG. 2, administration of combinations of PEG-rMuIL-10 and rMuIL-12 resulted in a larger reduction in tumor weight than that observed following administration of either agent alone. These data represent the beneficial anti-tumor effects of combination therapy.

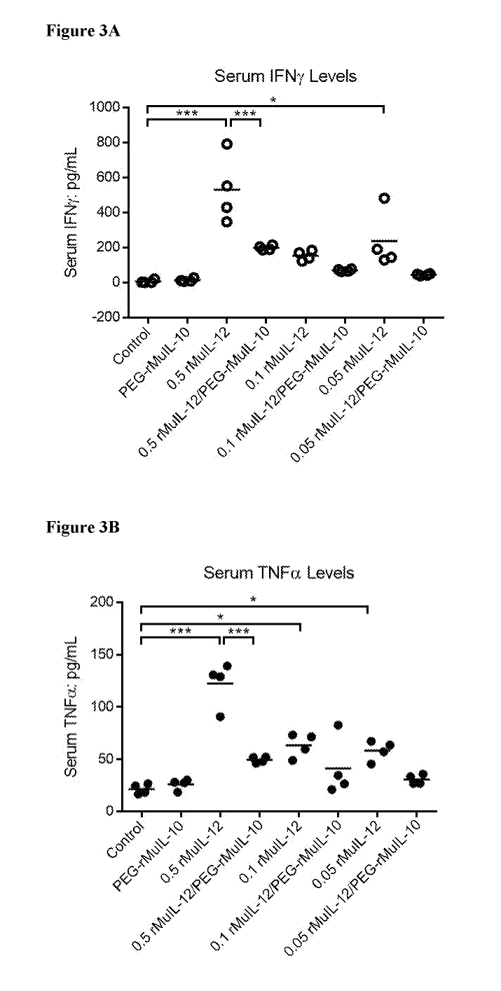

Next, the serum levels IFNγ and TNFα induced by PEG-rMuIL-10 and rMuIL-12, either alone or in combination, in 4T1 tumor bearing mice were evaluated. The data indicate that exposure to both PEG-rMuIL-10 and rMuIL-12 individually lead to the induction of serum IFNγ and TNFα. Surprisingly, when combined, administration of IL-12 and PEG-rMuIL-10 resulted in lower IFNγ (FIG. 3A) and TNFα (FIG. 3B) serum levels. In particular, combined exposure of IL-12 and PEG-rMuIL-10 exhibited significantly lower serum IFNγ than IL-12 alone. A particular embodiment of the present disclosure comprises the use of PEG-IL-10 to lower the serum cytokine (IFNγ and TNFα) levels induced by IL-12 treatment in order to detoxify IL-12, while simultaneously enhancing the anti-tumor benefits of administering the two agents together.

As noted above, while recent reports relating to combinations of IL-10 and IL-12 in the immunoncology setting have reported potential synergistic anti-tumor function, there is no disclosure of the potential control of IL-12—mediated toxicity by such combination therapy. Therefore, the data and other findings reported herein that the combination of IL-12 and PEG-IL-10 results in control of IL-12—associated toxicity, as indicated by a significant decrease in serum IFNγ (relative to that observed following IL-12 monotherapy) was both surprising and unexpected.

### Serum Concentrations

The blood plasma levels of IL-10 in the methods described herein can be characterized in several manners, including: (1) a mean IL-10 serum trough concentration above some specified level or in a range of levels; (2) a mean IL-10 serum trough concentration above some specified level for some amount of time; (3) a steady state IL-10 serum concentration level above or below some specified level or in a range of levels; or (4) a Cₘₐₓ of the concentration profile above or below some specified level or in some range of levels. As set forth herein, mean serum trough IL-10 concentrations have been found to be of particular import for efficacy in certain indications. Blood plasma levels of IL-12 can be characterized in a similar manner.

As set forth above, the desired IL-10 serum trough concentration may depend on a number of factors, including the nature of the disease, disorder or condition (e.g., localized tumor or metastatic disease), the extent to which the subject is suffering from the malady (e.g., early versus late stage disease), whether combination therapy is being administered, and patient-specific parameters (e.g., hepatic and renal function). By way of example, co-administration of PEG-IL-10 and a chemotherapeutic agent may only require a serum trough in the ˜1-2 ng/mL range in order to observe clinical benefit, while metastatic cancer may require 6-10 ng/mL or more to achieve comparable clinical benefit.

In particular embodiments of the present disclosure, the mean IL-10 serum trough concentration is at least 6.0 ng/mL, at least 7.0 ng/mL, at least 8.0 ng/mL, and least 9.0 ng/mL, at least 10.0 ng/mL, at least 11.0 ng/mL, at least 12.0 ng/mL, at least 13.0 ng/mL, at least 14.0 ng/mL, at least 15.0 ng/mL, at least 16.0 ng/mL, at least 17.0 ng/mL, at least 18.0 ng/mL, at least 19.0 ng/mL, at least 20.0 ng/mL, at least 21.0 ng/mL, at least 22.0 ng/mL, or greater than 22.0 ng/mL.

In other particular embodiments, the mean IL-10 serum trough concentration is at least 1.0 ng/mL, at least 1.5 ng/mL, at least 2.0 ng/mL, at least 2.5 ng/mL, at least 3.0 ng/mL, at least 3.5 ng/mL, at least 4.0 ng/mL, at least 4.5 ng/mL, at least 5.0 ng/mL, and least 5.5 ng/mL, at least 6.0 ng/mL, at least 6.5 ng/mL or greater than 7 ng/mL.

In further embodiments, the period of time is at least 12 hours, at least 24 hours, at least 48 hours, at least 72 hours, at least 1 week, at least 2 weeks, at least 3 weeks, at least 1 month, at least 6 weeks, at least 2 months, at least 3 months, or greater than 3 months.

In particular embodiments of the present disclosure, the mean IL-10 serum trough concentration is maintained for at least 85% of the period of time, at least 90%, at least 95%, at least 98%, at least 99% or 100% of the period of time.

In still further embodiments of the present disclosure, blood plasma and/or serum level concentration profiles that can be produced include: a mean IL-10 plasma and/or serum trough concentration of greater than about 1.0 pg/mL, greater than about 10.0 pg/mL, greater than about 20.0 pg/mL, greater than about 30 pg/mL, greater than about 40 pg/mL, greater than about 50.0 pg/mL, greater than about 60.0 pg/mL, greater than about 70.0 pg/mL, greater than about 80.0 pg/mL, greater than about 90 pg/mL, greater than about 0.1 ng/mL, greater than about 0.2 ng/mL, greater than about 0.3 ng/mL, greater than about 0.4 ng/mL, greater than about 0.5 ng/mL, greater than about 0.6 ng/mL, greater than about 0.7 ng/mL, greater than about 0.8 ng/mL, greater than about 0.9 ng/mL, greater than about 1.0 ng/mL, greater than about 1.5 ng/mL, greater than about 2.0 ng/mL, greater than about 2.5 ng/mL, greater than about 3.0 ng/mL, greater than about 3.5 ng/mL, greater than about 4.0 ng/mL, greater than about 4.5 ng/mL, greater than about 5.0 ng/mL, greater than about 5.5 ng/mL, greater than about 6.0 ng/mL, greater than about 6.5 ng/mL, greater than about 7.0 ng/mL, greater than about 7.5 ng/mL, greater than about 8.0 ng/mL, greater than about 8.5 ng/mL, greater than about 9.0 ng/mL, greater than about 9.5 ng/mL, or greater than about 10.0 ng/mL.

In particular embodiments of the present disclosure, a mean IL-10 serum trough concentration is in the range of from 1.0 pg/mL to 10 ng/mL. In some embodiments, the mean IL-10 serum trough concentration is in the range of from 1.0 pg/mL to 100 pg/mL. In other embodiments, the mean IL-10 serum trough concentration is in the range of from 0.1 ng/mL to 1.0 ng/mL. In still other embodiments, the mean IL-10 serum trough concentration is in the range of from 1.0 ng/mL to 10 ng/mL. It is to be understood that the present disclosure contemplates ranges incorporating any concentrations encompassed by those set forth herein even if such ranges are not explicitly recited. By way of example, the mean serum IL-10 concentration in an embodiment can be in the range of from 0.5 ng/mL to 5 ng/mL. By way of further examples, particular embodiments of the present disclosure comprise a mean IL-10 serum trough concentration in a range of from about 0.5 ng/mL to about 10.5 ng/mL, from about 1.0 ng/mL to about 10.0 ng/mL, from about 1.0 ng/mL to about 9.0 ng/mL, from about 1.0 ng/mL to about 8.0 ng/mL, from about 1.0 ng/mL to about 7.0 ng/mL, from about 1.5 ng/mL to about 10.0 ng/mL, from about 1.5 ng/mL to about 9.0 ng/mL, from about 1.5 ng/mL to about 8.0 ng/mL, from about 1.5 ng/mL to about 7.0 ng/mL, from about 2.0 ng/mL to about 10.0 ng/mL, from about 2.0 ng/mL to about 9.0 ng/mL, from about 2.0 ng/mL to about 8.0 ng/mL, and from about 2.0 ng/mL to about 7.0 ng/mL.

In particular embodiments, a mean IL-10 serum trough concentration of 1-2 ng/mL is maintained over the duration of treatment. The present disclosure also contemplates embodiments wherein the mean IL-10 serum peak concentration is less than or equal to about 10.0 ng/mL over the duration of treatment. Further embodiments contemplate a mean IL-10 serum trough concentration greater than or equal to about 10.0 ng/mL. The optimal mean serum concentration is generally that at which the desired therapeutic effect is achieved without introducing undesired adverse effects.

Certain embodiments of the present disclosure provide a method for monitoring a subject receiving IL-10 therapy to predict, and thus potentially avoid, adverse effects, the method comprising: (1) measuring the subject's peak concentration of IL-10; (2) measuring the subject's trough concentration of IL-10; (3) calculating a peak-trough fluctuation; and, (4) using the calculated peak-trough fluctuation to predict potential adverse effects in the subject. In particular subject populations, a smaller peak-trough fluctuation indicates a lower probability that the subject will experience IL-10—related adverse effects. In addition, in some embodiments particular peak-trough fluctuations are determined for the treatment of particular diseases, disorders and conditions using particular dosing parameters, and those fluctuations are used as reference standards.

For the majority of drugs, plasma drug concentrations decline in a multi-exponential fashion. Immediately after intravenous administration, the drug rapidly distributes throughout an initial space (minimally defined as the plasma volume), and then a slower, equilibrative distribution to extravascular spaces (e.g., certain tissues) occurs. Intravenous IL-10 administration is associated with such a two-compartment kinetic model (see Rachmawati, H. et al. (2004) Pharm. Res. 21(11):2072-78). The pharmacokinetics of subcutaneous recombinant hIL-10 has also been studied (Radwanski, E. et al. (1998) Pharm. Res. 15(12):1895-1901). Thus, volume-of-distribution considerations are pertinent when assessing appropriate IL-10 dosing-related parameters. Moreover, efforts to target IL-10 agents to specific cell types have been explored (see, e.g., Rachmawati, H. (May 2007) Drug Met. Dist. 35(5):814-21), and the leveraging of IL-10 pharmacokinetic and dosing principles can prove invaluable to the success of such efforts.

The present disclosure contemplates administration of any dose and dosing regimen that results in maintenance of any of the IL-10 serum trough concentrations set forth above. By way of example, but not limitation, when the subject is a human, non-pegylated hIL-10 can be administered at a dose greater than 0.5 μg/kg/day, greater than 1.0 μg/kg/day, greater than 2.5 μg/kg/day, greater than 5 μg/kg/day, greater than 7.5 μg/kg, greater than 10.0 μg/kg, greater than 12.5 μg/kg, greater than 15 μg/kg/day, greater than 17.5 μg/kg/day, greater than 20 μg/kg/day, greater than 22.5 μg/kg/day, greater than 25 μg/kg/day, greater than 30 μg/kg/day, or greater than 35 μg/kg/day. In addition, by way of example, but not limitation, when the subject is a human, pegylated hIL-10 comprising a relatively small PEG (e.g., 5 kDa mono-di-PEG-hIL-10) can be administered at a dose greater than 0.5 μg/kg/day, greater than 0.75 μg/kg/day, greater than 1.0 μg/kg/day, greater than 1.25 μg/kg/day, greater than 1.5 μg/kg/day, greater than 1.75 μg/kg/day, greater than 2.0 μg/kg/day, greater than 2.25 μg/kg/day, greater than 2.5 μg/kg/day, greater than 2.75 μg/kg/day, greater than 3.0 μg/kg/day, greater than 3.25 μg/kg/day, greater than 3.5 μg/kg/day, greater than 3.75 μg/kg/day, greater than 4.0 μg/kg/day, greater than 4.25 μg/kg/day, greater than 4.5 μg/kg/day, greater than 4.75 μg/kg/day, or greater than 5.0 μg/kg/day.

The skilled artisan (e.g., a pharmacologist) is able to determine the optimum dosing regimen(s) when a PEG-IL-10 is administered in combination with an IL-12 agent. By way of example, in some embodiments the optimum PEG-IL-10 dosing regimen may require a reduction in the amount of PEG-IL-10 administered per dose (e.g., less than 1.0 μg/kg/day, less than 0.75 μg/kg/day, less than 0.5 μg/kg/day, less than 0.25 μg/kg/day, or less than 0.125 μg/kg/day). In certain exemplary embodiments of the present disclosure, a mean IL-10 serum trough concentration may be in a range of from about 0.1 ng/mL to about 9.5 ng/mL, from about 0.25 ng/mL to about 8.0 ng/mL, from about 0.5 ng/mL to about 7.0 ng/mL, from about 0.75 ng/mL to about 6.0 ng/mL, or from about 1.0 ng/mL to about 5.0 ng/mL.

The present disclosure contemplates dosing an IL-12 agent such that the serum concentration achieves a peak and is then cleared such that it is essentially unmeasurable before it is administered again. By way of example, when a PEG-IL-10 is administered every 24 hours to maintain a serum trough concentration of ˜10 ng/mL, an IL-12 agent can be co-administered in an amount (e.g., 5 μg/kg/day) that results in a peak less that its MTD and then is metabolized such that there is no measurable serum level at the end of a 24-hour dosing cycle. As with administration of a PEG-IL-10, the dose of an IL-12 agent may depend on a number of factors, including the nature of the disease, disorder or condition (e.g., localized tumor or metastatic disease), the extent to which the subject is suffering from the malady (e.g., early versus late stage disease), whether combination therapy is being administered, and patient-specific parameters (e.g., hepatic and renal function).

When a PEG-IL-10 is administered in combination with an IL-12 agent such as those described herein, one or more of the dosing parameters of the PEG-IL-10 applicable to monotherapy can be modified while the dosing parameters of the IL-12 agent applicable to monotherapy can remain the same; one or more of the dosing parameters of the PEG-IL-10 applicable to monotherapy can remain the same while one or more of the dosing parameters of the IL-12 agent applicable to monotherapy can be modified; one or more of the dosing parameters of the PEG-IL-10 and the IL-12 agent applicable to monotherapy can be modified; or the dosing parameters of each of the PEG-IL-10 and the IL-12 agent can remain the same.

### Methods of Production of IL-10

A polypeptide of the present disclosure can be produced by any suitable method, including non-recombinant (e.g., chemical synthesis) and recombinant methods.

A. Chemical Synthesis

Where a polypeptide is chemically synthesized, the synthesis can proceed via liquid-phase or solid-phase. Solid-phase peptide synthesis (SPPS) allows the incorporation of unnatural amino acids and/or peptide/protein backbone modification. Various forms of SPPS, such as 9-fluorenylmethoxycarbonyl (Fmoc) and t-butyloxycarbonyl (Boc), are available for synthesizing polypeptides of the present disclosure. Details of the chemical syntheses are known in the art (e.g., Ganesan A. (2006) Mini Rev. Med. Chem. 6:3-10; and Camarero J. A. et al., (2005) Protein Pept Lett. 12:723-8).

Solid phase peptide synthesis can be performed as described hereafter. The alpha functions (Nα) and any reactive side chains are protected with acid-labile or base-labile groups. The protective groups are stable under the conditions for linking amide bonds but can readily be cleaved without impairing the peptide chain that has formed. Suitable protective groups for the α-amino function include, but are not limited to, the following: Boc, benzyloxycarbonyl (Z), O-chlorbenzyloxycarbonyl, bi-phenylisopropyloxycarbonyl, tert-amyloxycarbonyl (Amoc), α, α-dimethyl-3,5-dimethoxy-benzyloxycarbonyl, o-nitrosulfenyl, 2-cyano-t-butoxy-carbonyl, Fmoc, 1-(4,4-dimethyl-2,6-dioxocylohex-1-ylidene)ethyl (Dde) and the like.

Suitable side chain protective groups include, but are not limited to: acetyl, allyl (All), allyloxycarbonyl (Alloc), benzyl (Bzl), benzyloxycarbonyl (Z), t-butyloxycarbonyl (Boc), benzyloxymethyl (Bom), o-bromobenzyloxycarbonyl, t-butyl (tBu), t-butyldimethylsilyl, 2-chlorobenzyl, 2-chlorobenzyloxycarbonyl, 2,6-dichlorobenzyl, cyclohexyl, cyclopentyl, dimethyl-2,6-dioxocyclohex-1-ylidene)ethyl (Dde), isopropyl, 4-methoxy-2,3-6-trimethylbenzylsulfonyl (Mtr), 2,3,5,7,8-pentamethylchroman-6-sulfonyl (Pmc), pivalyl, tetrahydropyran-2-yl, tosyl (Tos), 2,4,6-trimethoxybenzyl, trimethylsilyl and trityl (Trt).

In the solid phase synthesis, the C-terminal amino acid is coupled to a suitable support material. Suitable support materials are those which are inert towards the reagents and reaction conditions for the step-wise condensation and cleavage reactions of the synthesis process and which do not dissolve in the reaction media being used. Examples of commercially-available support materials include styrene/divinylbenzene copolymers which have been modified with reactive groups and/or polyethylene glycol; chloromethylated styrene/divinylbenzene copolymers; hydroxymethylated or aminomethylated styrene/divinylbenzene copolymers; and the like. When preparation of the peptidic acid is desired, polystyrene (1%)-divinylbenzene or TentaGel® derivatized with 4-benzyloxybenzyl-alcohol (Wang-anchor) or 2-chlorotrityl chloride can be used. In the case of the peptide amide, polystyrene (1%) divinylbenzene or TentaGel® derivatized with 5-(4′-aminomethyl)-3′,5′-dimethoxyphenoxy)valeric acid (PAL-anchor) or p-(2,4-dimethoxyphenyl-amino methyl)-phenoxy group (Rink amide anchor) can be used.

The linkage to the polymeric support can be achieved by reacting the C-terminal Fmoc-protected amino acid with the support material by the addition of an activation reagent in ethanol, acetonitrile, N,N-dimethylformamide (DMF), dichloromethane, tetrahydrofuran, N-methylpyrrolidone or similar solvents at room temperature or elevated temperatures (e.g., between 40° C. and 60° C.) and with reaction times of, e.g., 2 to 72 hours.

The coupling of the Nα-protected amino acid (e.g., the Fmoc amino acid) to the PAL, Wang or Rink anchor can, for example, be carried out with the aid of coupling reagents such as N,N′-dicyclohexylcarbodiimide (DCC), N,N′-diisopropylcarbodiimide (DIC) or other carbodiimides, 2-(1H-benzotriazol-1-yl)-1,1,3,3-tetramethyluronium tetrafluoroborate (TBTU) or other uronium salts, 0-acyl-ureas, benzotriazol-1-yl-tris-pyrrolidino-phosphonium hexafluorophosphate (PyBOP) or other phosphonium salts, N-hydroxysuccinimides, other N-hydroxyimides or oximes in the presence or absence of 1-hydroxybenzotriazole or 1-hydroxy-7-azabenzotriazole, e.g., with the aid of TBTU with addition of HOBt, with or without the addition of a base such as, for example, diisopropylethylamine (DIEA), triethylamine or N-methylmorpholine, e.g., diisopropylethylamine with reaction times of 2 to 72 hours (e.g., 3 hours in a 1.5 to 3-fold excess of the amino acid and the coupling reagents, for example, in a 2-fold excess and at temperatures between about 10° C. and 50° C., for example, 25° C. in a solvent such as dimethylformamide, N-methylpyrrolidone or dichloromethane, e.g., dimethylformamide).

Instead of the coupling reagents, it is also possible to use the active esters (e.g., pentafluorophenyl, p-nitrophenyl or the like), the symmetric anhydride of the Nα-Fmoc-amino acid, its acid chloride or acid fluoride, under the conditions described above.

The Nα-protected amino acid (e.g., the Fmoc amino acid) can be coupled to the 2-chlorotrityl resin in dichloromethane with the addition of DIEA and having reaction times of 10 to 120 minutes, e.g., 20 minutes, but is not limited to the use of this solvent and this base.

The successive coupling of the protected amino acids can be carried out according to conventional methods in peptide synthesis, typically in an automated peptide synthesizer. After cleavage of the Nα-Fmoc protective group of the coupled amino acid on the solid phase by treatment with, e.g., piperidine (10% to 50%) in dimethylformamide for 5 to 20 minutes, e.g., 2×2 minutes with 50% piperidine in DMF and 1×15 minutes with 20% piperidine in DMF, the next protected amino acid in a 3 to 10-fold excess, e.g., in a 10-fold excess, is coupled to the previous amino acid in an inert, non-aqueous, polar solvent such as dichloromethane, DMF or mixtures of the two and at temperatures between about 10° C. and 50° C., e.g., at 25° C. The previously mentioned reagents for coupling the first Nα-Fmoc amino acid to the PAL, Wang or Rink anchor are suitable as coupling reagents. Active esters of the protected amino acid, or chlorides or fluorides or symmetric anhydrides thereof can also be used as an alternative.

At the end of the solid phase synthesis, the peptide is cleaved from the support material while simultaneously cleaving the side chain protecting groups. Cleavage can be carried out with trifluoroacetic acid or other strongly acidic media with addition of 5%-20% V/V of scavengers such as dimethyl sulfide, ethylmethylsulfide, thioanisole, thiocresol, m-cresol, anisole ethanedithiol, phenol or water, e.g., 15% v/v dimethylsulfide/ethanedithiol/m-cresol 1:1:1, within 0.5 to 3 hours, e.g., 2 hours. Peptides with fully protected side chains are obtained by cleaving the 2-chlorotrityl anchor with glacial acetic acid/trifluoroethanol/dichloromethane 2:2:6. The protected peptide can be purified by chromatography on silica gel. If the peptide is linked to the solid phase via the Wang anchor and if it is intended to obtain a peptide with a C-terminal alkylamidation, the cleavage can be carried out by aminolysis with an alkylamine or fluoroalkylamine. The aminolysis is carried out at temperatures between about −10° C. and 50° C. (e.g., about 25° C.), and reaction times between about 12 and 24 hours (e.g., about 18 hours). In addition, the peptide can be cleaved from the support by re-esterification, e.g., with methanol.

The acidic solution that is obtained can be admixed with a 3 to 20-fold amount of cold ether or n-hexane, e.g., a 10-fold excess of diethyl ether, in order to precipitate the peptide and hence to separate the scavengers and cleaved protective groups that remain in the ether. A further purification can be carried out by re-precipitating the peptide several times from glacial acetic acid. The precipitate that is obtained can be taken up in water or tert-butanol or mixtures of the two solvents, e.g., a 1:1 mixture of tert-butanol/water, and freeze-dried.

The peptide obtained can be purified by various chromatographic methods, including ion exchange over a weakly basic resin in the acetate form; hydrophobic adsorption chromatography on non-derivatized polystyrene/divinylbenzene copolymers (e.g., Amberlite® XAD); adsorption chromatography on silica gel; ion exchange chromatography, e.g., on carboxymethyl cellulose; distribution chromatography, e.g., on Sephadex® G-25; countercurrent distribution chromatography; or high pressure liquid chromatography (HPLC) e.g., reversed-phase HPLC on octyl or octadecylsilylsilica (ODS) phases.

B. Recombinant Production

Methods describing the preparation of human and mouse IL-10 can be found in, for example, U.S. Pat. No. 5,231,012, which teaches methods for the production of proteins having IL-10 activity, including recombinant and other synthetic techniques. IL-10 can be of viral origin, and the cloning and expression of a viral IL-10 from Epstein Barr virus (BCRF1 protein) is disclosed in Moore et al., (1990) Science 248:1230. IL-10 can be obtained in a number of ways using standard techniques known in the art, such as those described herein. Recombinant human IL-10 is also commercially available, e.g., from PeproTech, Inc., Rocky Hill, N.J.

Where a polypeptide is produced using recombinant techniques, the polypeptide can be produced as an intracellular protein or as a secreted protein, using any suitable construct and any suitable host cell, which can be a prokaryotic or eukaryotic cell, such as a bacterial (e.g., E. coli) or a yeast host cell, respectively. Other examples of eukaryotic cells that can be used as host cells include insect cells, mammalian cells, and/or plant cells. Where mammalian host cells are used, they can include human cells (e.g., HeLa, 293, H9 and Jurkat cells); mouse cells (e.g., NIH3T3, L cells, and C127 cells); primate cells (e.g., Cos 1, Cos 7 and CV1); and hamster cells (e.g., Chinese hamster ovary (CHO) cells).

A variety of host-vector systems suitable for the expression of a polypeptide can be employed according to standard procedures known in the art. See, e.g., Sambrook et al., 1989 Current Protocols in Molecular Biology Cold Spring Harbor Press, New York; and Ausubel et al. 1995 Current Protocols in Molecular Biology, Eds. Wiley and Sons. Methods for introduction of genetic material into host cells include, for example, transformation, electroporation, conjugation, calcium phosphate methods and the like. The method for transfer can be selected so as to provide for stable expression of the introduced polypeptide-encoding nucleic acid. The polypeptide-encoding nucleic acid can be provided as an inheritable episomal element (e.g., a plasmid) or can be genomically integrated. A variety of appropriate vectors for use in production of a polypeptide of interest are commercially available.

Vectors can provide for extrachromosomal maintenance in a host cell or can provide for integration into the host cell genome. The expression vector provides transcriptional and translational regulatory sequences, and can provide for inducible or constitutive expression where the coding region is operably-linked under the transcriptional control of the transcriptional initiation region, and a transcriptional and translational termination region. In general, the transcriptional and translational regulatory sequences can include, but are not limited to, promoter sequences, ribosomal binding sites, transcriptional start and stop sequences, translational start and stop sequences, and enhancer or activator sequences. Promoters can be either constitutive or inducible, and can be a strong constitutive promoter (e.g., T7).

Expression constructs generally have convenient restriction sites located near the promoter sequence to provide for the insertion of nucleic acid sequences encoding proteins of interest. A selectable marker operative in the expression host can be present to facilitate selection of cells containing the vector. Moreover, the expression construct can include additional elements. For example, the expression vector can have one or two replication systems, thus allowing it to be maintained in organisms, for example, in mammalian or insect cells for expression and in a prokaryotic host for cloning and amplification. In addition, the expression construct can contain a selectable marker gene to allow the selection of transformed host cells. Selectable genes are well known in the art and will vary with the host cell used.

Isolation and purification of a protein can be accomplished according to methods known in the art. For example, a protein can be isolated from a lysate of cells genetically modified to express the protein constitutively and/or upon induction, or from a synthetic reaction mixture by immunoaffinity purification, which generally involves contacting the sample with an anti-protein antibody, washing to remove non-specifically bound material, and eluting the specifically bound protein. The isolated protein can be further purified by dialysis and other methods normally employed in protein purification. In one embodiment, the protein can be isolated using metal chelate chromatography methods. Proteins can contain modifications to facilitate isolation.

The polypeptides can be prepared in substantially pure or isolated form (e.g., free from other polypeptides). The polypeptides can be present in a composition that is enriched for the polypeptide relative to other components that can be present (e.g., other polypeptides or other host cell components). For example, purified polypeptide can be provided such that the polypeptide is present in a composition that is substantially free of other expressed proteins, e.g., less than about 90%, less than about 60%, less than about 50%, less than about 40%, less than about 30%, less than about 20%, less than about 10%, less than about 5%, or less than about 1%.

An IL-10 polypeptide can be generated using recombinant techniques to manipulate different IL-10—related nucleic acids known in the art to provide constructs capable of encoding the IL-10 polypeptide. It will be appreciated that, when provided a particular amino acid sequence, the ordinary skilled artisan will recognize a variety of different nucleic acid molecules encoding such amino acid sequence in view of her background and experience in, for example, molecular biology.

### Amide Bond Substitutions

In some cases, IL-10 includes one or more linkages other than peptide bonds, e.g., at least two adjacent amino acids are joined via a linkage other than an amide bond. For example, in order to reduce or eliminate undesired proteolysis or other means of degradation, and/or to increase serum stability, and/or to restrict or increase conformational flexibility, one or more amide bonds within the backbone of IL-10 can be substituted.

In another example, one or more amide linkages (—CO—NH—) in IL-10 can be replaced with a linkage which is an isostere of an amide linkage, such as —CH₂NH—, —CH₂S—, —CH₂CH₂—, —CH═CH-(cis and trans), —COCH₂—, —CH(OH)CH₂— or —CH₂SO—. One or more amide linkages in IL-10 can also be replaced by, for example, a reduced isostere pseudopeptide bond. See Couder et al. (1993) Int. J. Peptide Protein Res. 41:181-184. Such replacements and how to effect them are known to those of ordinary skill in the art.

### Amino Acid Substitutions

One or more amino acid substitutions can be made in an IL-10 polypeptide. The following are non-limiting examples:

a) substitution of alkyl-substituted hydrophobic amino acids, including alanine, leucine, isoleucine, valine, norleucine, (S)-2-aminobutyric acid, (S)-cyclohexylalanine or other simple alpha-amino acids substituted by an aliphatic side chain from C₁-C₁₀ carbons including branched, cyclic and straight chain alkyl, alkenyl or alkynyl substitutions;

b) substitution of aromatic-substituted hydrophobic amino acids, including phenylalanine, tryptophan, tyrosine, sulfotyrosine, biphenylalanine, 1-naphthylalanine, 2-naphthylalanine, 2-benzothienylalanine, 3-benzothienylalanine, histidine, including amino, alkylamino, dialkylamino, aza, halogenated (fluoro, chloro, bromo, or iodo) or alkoxy (from C₁-C₄)-substituted forms of the above-listed aromatic amino acids, illustrative examples of which are: 2-, 3- or 4-aminophenylalanine, 2-, 3- or 4-chlorophenylalanine, 2-, 3- or 4-methylphenylalanine, 2-, 3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2, 3, or 4-biphenylalanine, 2′-, 3′-, or 4′-methyl-, 2-, 3- or 4-biphenylalanine, and 2- or 3-pyridylalanine;

c) substitution of amino acids containing basic side chains, including arginine, lysine, histidine, ornithine, 2,3-diaminopropionic acid, homoarginine, including alkyl, alkenyl, or aryl-substituted (from C₁-C₁₀ branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma'-diethyl-homoarginine. Included also are compounds such as alpha-methyl-arginine, alpha-methyl-2,3-diaminopropionic acid, alpha-methyl-histidine, alpha-methyl-ornithine where the alkyl group occupies the pro-R position of the alpha-carbon. Also included are the amides formed from alkyl, aromatic, heteroaromatic (where the heteroaromatic group has one or more nitrogens, oxygens or sulfur atoms singly or in combination), carboxylic acids or any of the many well-known activated derivatives such as acid chlorides, active esters, active azolides and related derivatives, and lysine, ornithine, or 2,3-diaminopropionic acid;

d) substitution of acidic amino acids, including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids;

e) substitution of side chain amide residues, including asparagine, glutamine, and alkyl or aromatic substituted derivatives of asparagine or glutamine; and

f) substitution of hydroxyl-containing amino acids, including serine, threonine, homoserine, 2,3-diaminopropionic acid, and alkyl or aromatic substituted derivatives of serine or threonine.

In some cases, IL-10 comprises one or more naturally occurring non-genetically encoded L-amino acids, synthetic L-amino acids, or D-enantiomers of an amino acid. For example, IL-10 can comprise only D-amino acids. For example, an IL-10 polypeptide can comprise one or more of the following residues: hydroxyproline, β-alanine, o-aminobenzoic acid, m-aminobenzoic acid, p-aminobenzoic acid, m-aminomethylbenzoic acid, 2,3-diaminopropionic acid, α-aminoisobutyric acid, N-methylglycine (sarcosine), ornithine, citrulline, t-butylalanine, t-butylglycine, N-methylisoleucine, phenylglycine, cyclohexylalanine, norleucine, naphthylalanine, pyridylalanine 3-benzothienyl alanine, 4-chlorophenylalanine, 2-fluorophenylalanine, 3-fluorophenylalanine, 4-fluorophenylalanine, penicillamine, 1,2,3,4-tetrahydroisoquinoline-3-carboxylic acid, β-2-thienylalanine, methionine sulfoxide, homoarginine, N-acetyl lysine, 2,4-diamino butyric acid, rho-aminophenylalanine, N-methylvaline, homocysteine, homoserine, ε-amino hexanoic acid, ω-aminohexanoic acid, ω-aminoheptanoic acid, ω-aminooctanoic acid, ω-aminodecanoic acid, ω-aminotetradecanoic acid, cyclohexylalanine, α,γ-diaminobutyric acid, α,β-diaminopropionic acid, δ-amino valeric acid, and 2,3-diaminobutyric acid.

### Additional Modifications

A cysteine residue or a cysteine analog can be introduced into an IL-10 polypeptide to provide for linkage to another peptide via a disulfide linkage or to provide for cyclization of the IL-10 polypeptide. Methods of introducing a cysteine or cysteine analog are known in the art; see, e.g., U.S. Pat. No. 8,067,532.

An IL-10 polypeptide can be cyclized. One or more cysteines or cysteine analogs can be introduced into an IL-10 polypeptide, where the introduced cysteine or cysteine analog can form a disulfide bond with a second introduced cysteine or cysteine analog. Other means of cyclization include introduction of an oxime linker or a lanthionine linker; see, e.g., U.S. Pat. No. 8,044,175. Any combination of amino acids (or non-amino acid moieties) that can form a cyclizing bond can be used and/or introduced. A cyclizing bond can be generated with any combination of amino acids (or with an amino acid and —(CH2)ₙ—CO— or —(CH2)ₙ—C₆H₄—CO—) with functional groups which allow for the introduction of a bridge. Some examples are disulfides, disulfide mimetics such as the —(CH2)ₙ— carba bridge, thioacetal, thioether bridges (cystathionine or lanthionine) and bridges containing esters and ethers. In these examples, n can be any integer, but is frequently less than ten.

Other modifications include, for example, an N-alkyl (or aryl) substitution (ψ[CONR]), or backbone crosslinking to construct lactams and other cyclic structures. Other derivatives include C-terminal hydroxymethyl derivatives, o-modified derivatives (e.g., C-terminal hydroxymethyl benzyl ether), N-terminally modified derivatives including substituted amides such as alkylamides and hydrazides.

In some cases, one or more L-amino acids in an IL-10 polypeptide is replaced with one or more D-amino acids.

In some cases, an IL-10 polypeptide is a retroinverso analog (see, e.g., Sela and Zisman (1997) FASEB J. 11:449). Retro-inverso peptide analogs are isomers of linear polypeptides in which the direction of the amino acid sequence is reversed (retro) and the chirality, D- or L-, of one or more amino acids therein is inverted (inverso), e.g., using D-amino acids rather than L-amino acids. (See, e.g., Jameson et al. (1994) Nature 368:744; and Brady et al. (1994) Nature 368:6920.

An IL-10 polypeptide can include a “Protein Transduction Domain” (PTD), which refers to a polypeptide, polynucleotide, carbohydrate, or organic or inorganic molecule that facilitates traversing a lipid bilayer, micelle, cell membrane, organelle membrane, or vesicle membrane. A PTD attached to another molecule facilitates the molecule traversing a membrane, for example going from extracellular space to intracellular space, or cytosol to within an organelle. In some embodiments, a PTD is covalently linked to the amino terminus of an IL-10 polypeptide, while in other embodiments, a PTD is covalently linked to the carboxyl terminus of an IL-10 polypeptide. Exemplary protein transduction domains include, but are not limited to, a minimal undecapeptide protein transduction domain (corresponding to residues 47-57 of HIV-1 TAT comprising YGRKKRRQRRR; SEQ ID NO:3); a polyarginine sequence comprising a number of arginine residues sufficient to direct entry into a cell (e.g., 3, 4, 5, 6, 7, 8, 9, 10, or 10-50 arginines); a VP22 domain (Zender et al. (2002) Cancer Gene Ther. 9(6):489-96); a Drosophila Antennapedia protein transduction domain (Noguchi et al. (2003) Diabetes 52(7):1732-1737); a truncated human calcitonin peptide (Trehin et al. (2004) Pharm. Research 21:1248-1256); polylysine (Wender et al. (2000) Proc. Natl. Acad. Sci. USA 97:13003-13008); RRQRRTSKLMKR (SEQ ID NO:4); Transportan GWTLNSAGYLLGKINLKALAALAKKIL (SEQ ID NO:5); KALAWEAKLAKALAKALAKHLAKALAKALKCEA (SEQ ID NO:6); and RQIKIWFQNRRMKWKK (SEQ ID NO:7). Exemplary PTDs include, but are not limited to, YGRKKRRQRRR (SEQ ID NO:3), RKKRRQRRR (SEQ ID NO:8); an arginine homopolymer of from 3 arginine residues to 50 arginine residues; exemplary PTD domain amino acid sequences include, but are not limited to, any of the following: YGRKKRRQRRR (SEQ ID NO:3); RKKRRQRR (SEQ ID NO:9); YARAAARQARA (SEQ ID NO:10); THRLPRRRRRR (SEQ ID NO:11); and GGRRARRRRRR (SEQ ID NO:12).

The carboxyl group COR₃ of the amino acid at the C-terminal end of an IL-10 polypeptide can be present in a free form (R₃═OH) or in the form of a physiologically-tolerated alkaline or alkaline earth salt such as, e.g., a sodium, potassium or calcium salt. The carboxyl group can also be esterified with primary, secondary or tertiary alcohols such as, e.g., methanol, branched or unbranched C₁-C₆-alkyl alcohols, e.g., ethyl alcohol or tert-butanol. The carboxyl group can also be amidated with primary or secondary amines such as ammonia, branched or unbranched C₁-C₆-alkylamines or C₁-C₆ di-alkylamines, e.g., methylamine or dimethylamine.

The amino group of the amino acid NR₁R₂ at the N-terminus of an IL-10 polypeptide can be present in a free form (R₁═H and R₂═H) or in the form of a physiologically-tolerated salt such as, e.g., a chloride or acetate. The amino group can also be acetylated with acids such that R₁═H and R₂=acetyl, trifluoroacetyl, or adamantyl. The amino group can be present in a form protected by amino-protecting groups conventionally used in peptide chemistry, such as those provided above (e.g., Fmoc, Benzyloxy-carbonyl (Z), Boc, and Alloc). The amino group can be N-alkylated in which R₁ and/or R₂═C₁-C₆ alkyl or C₂-C₈ alkenyl or C₇-C₉ aralkyl. Alkyl residues can be straight-chained, branched or cyclic (e.g., ethyl, isopropyl and cyclohexyl, respectively).

### Pegylation of IL-10

Pegylation of IL-10 comprises conjugating or linking the IL-10 polypeptide sequence to any of a variety of nonproteinaceous polymers, e.g., polyethylene glycol (PEG), polypropylene glycol, or polyoxyalkylenes. This is frequently effected by a linking moiety covalently bound to both the protein and the nonproteinaceous polymer, e.g., a PEG. Such PEG-conjugated biomolecules have been shown to possess clinically useful properties, including better physical and thermal stability, protection against susceptibility to enzymatic degradation, increased solubility, longer in vivo circulating half-life and decreased clearance, reduced immunogenicity and antigenicity, and reduced toxicity. In addition to the beneficial effects of pegylation on pharmacokinetic parameters, pegylation itself can enhance activity. For example, PEG-IL-10 has been shown to be more efficacious against certain cancers than unpegylated IL-10 (see, e.g., EP 206636A2).

PEGs suitable for conjugation to a polypeptide sequence are generally soluble in water at room temperature, and have the general formula R(O—CH₂-CH₂)ₙO—R, where R is hydrogen or a protective group such as an alkyl or an alkanol group, and where n is an integer from 1 to 1000. When R is a protective group, it generally has from 1 to 8 carbons. The PEG conjugated to the polypeptide sequence can be linear or branched. Branched PEG derivatives, “star-PEGs” and multi-armed PEGs are contemplated by the present disclosure. A molecular weight of the PEG used in the present disclosure is not restricted to any particular range, and examples are set forth elsewhere herein; by way of example, certain embodiments have molecular weights between 5 kDa and 20 kDa, while other embodiments have molecular weights between 4 kDa and 10 kDa.

The present disclosure also contemplates compositions of conjugates wherein the PEGs have different n values, and thus the various different PEGs are present in specific ratios. For example, some compositions comprise a mixture of conjugates where n=1, 2, 3 and 4. In some compositions, the percentage of conjugates where n=1 is 18-25%, the percentage of conjugates where n=2 is 50-66%, the percentage of conjugates where n=3 is 12-16%, and the percentage of conjugates where n=4 is up to 5%. Such compositions can be produced by reaction conditions and purification methods know in the art. Exemplary reaction conditions are described throughout the specification. Cation exchange chromatography can be used to separate conjugates, and a fraction is then identified which contains the conjugate having, for example, the desired number of PEGs attached, purified free from unmodified protein sequences and from conjugates having other numbers of PEGs attached.

Pegylation most frequently occurs at the alpha amino group at the N-terminus of the polypeptide, the epsilon amino group on the side chain of lysine residues, and the imidazole group on the side chain of histidine residues. Since most recombinant polypeptides possess a single alpha and a number of epsilon amino and imidazole groups, numerous positional isomers can be generated depending on the linker chemistry. General pegylation strategies known in the art can be applied herein.

Two widely used first generation activated monomethoxy PEGs (mPEGs) are succinimdyl carbonate PEG (SC-PEG; see, e.g., Zalipsky, et al. (1992) Biotechnol. Appl. Biochem 15:100-114; and Miron and Wilcheck (1993) Bio-conjug. Chem. 4:568-569) and benzotriazole carbonate PEG (BTC-PEG; see, e.g., Dolence, et al. U.S. Pat. No. 5,650,234), which react preferentially with lysine residues to form a carbamate linkage, but are also known to react with histidine and tyrosine residues. The linkage to histidine residues on certain molecules (e.g., IFN-α) has been shown to be a hydrolytically unstable imidazolecarbamate linkage (see, e.g., Lee and McNemar, U.S. Pat. No. 5,985,263). Second generation pegylation technology has been designed to avoid these unstable linkages as well as the lack of selectivity in residue reactivity. Use of a PEG-aldehyde linker targets a single site on the N-terminus of a polypeptide through reductive amination.

PEG can be bound to a polypeptide of the present disclosure via a terminal reactive group (a “spacer”) which mediates a bond between the free amino or carboxyl groups of one or more of the polypeptide sequences and polyethylene glycol. The PEG having the spacer which can be bound to the free amino group includes N-hydroxysuccinylimide polyethylene glycol, which can be prepared by activating succinic acid ester of polyethylene glycol with N-hydroxysuccinylimide. Another activated polyethylene glycol which can be bound to a free amino group is 2,4-bis(O-methoxypolyethyleneglycol)-6-chloro-s-triazine, which can be prepared by reacting polyethylene glycol monomethyl ether with cyanuric chloride. The activated polyethylene glycol which is bound to the free carboxyl group includes polyoxyethylenediamine.

Conjugation of one or more of the polypeptide sequences of the present disclosure to PEG having a spacer can be carried out by various conventional methods. For example, the conjugation reaction can be carried out in solution at a pH of from 5 to 10, at temperature from 4° C. to room temperature, for 30 minutes to 20 hours, utilizing a molar ratio of reagent to protein of from 4:1 to 30:1. Reaction conditions can be selected to direct the reaction towards producing predominantly a desired degree of substitution. In general, low temperature, low pH (e.g., pH=5), and short reaction time tend to decrease the number of PEGs attached, whereas high temperature, neutral to high pH (e.g., pH>7), and longer reaction time tend to increase the number of PEGs attached. Various means known in the art can be used to terminate the reaction. In some embodiments the reaction is terminated by acidifying the reaction mixture and freezing at, e.g., −20° C. Pegylation of various molecules is discussed in, for example, U.S. Pat. Nos. 5,252,714; 5,643,575; 5,919,455; 5,932,462; and 5,985,263. PEG-IL-10 is described in, e.g., U.S. Pat. No. 7,052,686. Specific reaction conditions contemplated for use herein are set forth in the Experimental section.

The present disclosure also contemplates the use of PEG mimetics. Recombinant PEG mimetics have been developed that retain the attributes of PEG (e.g., enhanced serum half-life) while conferring several additional advantageous properties. By way of example, simple polypeptide chains (comprising, for example, Ala, Glu, Gly, Pro, Ser and Thr) capable of forming an extended conformation similar to PEG can be produced recombinantly already fused to the peptide or protein drug of interest (e.g., Amunix' XTEN technology; Mountain View, Calif.). This obviates the need for an additional conjugation step during the manufacturing process. Moreover, established molecular biology techniques enable control of the side chain composition of the polypeptide chains, allowing optimization of immunogenicity and manufacturing properties.

Linkers: Linkers and their use have been described above. Suitable linkers include “flexible linkers” which are generally of sufficient length to permit some movement between the modified polypeptide sequences and the linked components and molecules. The linker molecules are generally about 6-50 atoms long. The linker molecules may also be, for example, aryl acetylene, ethylene glycol oligomers containing 2-10 monomer units, diamines, diacids, amino acids, or combinations thereof. Suitable linkers can be readily selected and can be of any suitable length, such as 1 amino acid (e.g., Gly), 2, 3, 4, 5, 6, 7, 8, 9, 10, 10-20, 20-30, 30-50 or more than 50 amino acids.

Examples of flexible linkers include glycine polymers (G)ₙ, glycine-alanine polymers, alanine-serine polymers, glycine-serine polymers (for example, (GₘSₒ)ₙ, (GSGGS)ₙ (SEQ ID NO:13), (GₘSₒGₘ)ₙ, (GₘSₒGₘSₒGₘ)ₙ (SEQ ID NO:14), (GSGGSₘ)ₙ (SEQ ID NO:15), (GSGSₘG)ₙ (SEQ ID NO:16) and (GGGSₘ)ₙ (SEQ ID NO:17), and combinations thereof, where m, n, and o are each independently selected from an integer of at least 1 to 20, e.g., 1-18, 2-16, 3-14, 4-12, 5-10, 1, 2, 3, 4, 5, 6, 7, 8,9, or 10), and other flexible linkers. Glycine and glycine-serine polymers are relatively unstructured, and therefore may serve as a neutral tether between components. Examples of flexible linkers include, but are not limited to GGSG (SEQ ID NO:18), GGSGG (SEQ ID NO:19), GSGSG (SEQ ID NO:14), GSGGG (SEQ ID NO:20), GGGSG (SEQ ID NO:21), and GSSSG (SEQ ID NO:22).

Additional examples of flexible linkers include glycine polymers (G)n or glycine-serine polymers (e.g., (GS)n, (GSGGS)n (SEQ ID NO:13), (GGGS)n (SEQ ID NO:23) and (GGGGS)n (SEQ ID NO:24), where n=1 to 50, for example, 1, 2, 3, 4, 5, 6, 7, 8, 9, 10, 10-20, 20-30, 30-50). Exemplary flexible linkers include, but are not limited to GGGS (SEQ ID NO:23), GGGGS (SEQ ID NO:24), GGSG (SEQ ID NO:18), GGSGG (SEQ ID NO:19), GSGSG (SEQ ID NO:14), GSGGG (SEQ ID NO:20), GGGSG (SEQ ID NO:21), and GSSSG (SEQ ID NO:22). A multimer (e.g., 1, 2, 3, 4, 5, 6, 7, 8, 9, 10, 10-20, 20-30, or 30-50) of these linker sequences may be linked together to provide flexible linkers that may be used to conjugate a heterologous amino acid sequence to the polypeptides disclosed herein.

### Therapeutic and Prophylactic Uses

In particular embodiments, the present disclosure contemplates the use of a PEG-IL-10 and an IL-12 agent in the treatment and/or prevention of cancer-related diseases, disorders or conditions. While particular uses are described in detail hereafter, it is to be understood that the present disclosure is not so limited.

Representative cancers that may be treated or prevented using the combination therapies disclosed herein include cancer of the uterus, cervix, ovaries, breast, prostate, testes, gastrointestinal tract (e.g., esophagus, oropharynx, stomach, small or large intestines, colon, or rectum), kidney, renal cell, bladder, bone, bone marrow, skin, head or neck, liver, gall bladder, heart, lung, pancreas, salivary gland, adrenal gland, thyroid, brain (e.g., gliomas), ganglia, central nervous system (CNS) and peripheral nervous system (PNS), and cancers of the immune system (e.g., spleen or thymus).

The present disclosure also provides methods of treating or preventing other cancer-related diseases, disorders or conditions, including, for example, immunogenic tumors, non-immunogenic tumors, dormant tumors, virus-induced cancers (e.g., epithelial cell cancers, endothelial cell cancers, squamous cell carcinomas and papillomavirus), adenocarcinomas, lymphomas (e.g., a B-cell lymphoma), leukemias, carcinomas, melanomas, myelomas, sarcomas, teratocarcinomas, chemically-induced cancers, and metastasis. In particular embodiments, the tumor or cancer is colon cancer, ovarian cancer, breast cancer, melanoma, lung cancer, or glioblastoma.

In further particular embodiments, the cancer is mammary adenocarcinoma, lung alveolar carcinoma, fibrosarcoma, and pulmonary metastasis of melanoma (Pegram et al. (2012) Advancements in Tumor Immunotherapy and Cancer Vaccines, Dr. Hilal Arnouk (Ed.), ISBN: 978-953-307-998-1, InTech). Clinical studies exploring the antitumor effects of IL-12 based treatment in combination therapies or gene therapy include treatment of the following tumors: breast, pancreatic, hepatic, renal, cervical, gastrointestinal carcinomas, colorectal, Non-Hodgkin's lymphoma, melanoma (e.g., multiple melanoma), and AIDS-associated Kaposi sarcoma (Lasek et al. (2014) Cancer Immunol Immunother 63:419-35).

In particular embodiments of the present disclosure, the cancer-related disease, disorder or condition is an immune-insensitive tumor. Tumors that are insensitive to therapeutic immune manipulation may be described as exhibiting the following two characteristics: 1) active suppression of the immune system, and 2) an inflammatory response accompanied by the concomitant activation of immune-suppressive mechanisms resulting from treatment thereof (Galon et al. (Jul. 25 2013) Immunity 39:11-26 (PubMed PMID: 238900600). Examples of immune-insensitive tumors include, but are not limited to, colon, gastroesophageal, pancreatic and breast cancer.

As described elsewhere herein, in some embodiments the present disclosure provides methods for treating a cancer-related disease, disorder or condition with a PEG-IL-10 and an IL-12 agent in combination with at least one additional therapeutic or diagnostic agent, examples of which are provided herein.

### Pharmaceutical Compositions

The PEG-IL-10 and IL-12 agents contemplated by the present disclosure can be in the form of compositions suitable for administration to a subject. In general, such compositions are “pharmaceutical compositions” comprising PEG-IL-10 and/or an IL-12 agent and one or more pharmaceutically acceptable or physiologically acceptable diluents, carriers or excipients. In certain embodiments, the PEG-IL-10 and IL-12 agents are each present in a therapeutically acceptable amount. The pharmaceutical compositions can be used in the methods of the present disclosure; thus, for example, the pharmaceutical compositions can be administered ex vivo or in vivo to a subject in order to practice the therapeutic and prophylactic methods and uses described herein.

In the description of the pharmaceutical compositions, and aspects thereof, that follows, the pharmaceutical compositions are generally described in the context of a PEG-IL-10. However, it is to be understood that the description also applies to the IL-12 agents of the present disclosure, either in pharmaceutical compositions comprising combinations of a PEG-IL-10 and an IL-12 agent, or in pharmaceutical compositions comprising only one of the components.

The pharmaceutical compositions of the present disclosure can be formulated to be compatible with the intended method or route of administration; exemplary routes of administration are set forth herein. Furthermore, the pharmaceutical compositions can be used in combination with other therapeutically active agents or compounds as described herein in order to treat or prevent the diseases, disorders and conditions as contemplated by the present disclosure.

The pharmaceutical compositions typically comprise a therapeutically effective amount of a PEG-IL-10 and/or an IL-12 agent contemplated by the present disclosure and one or more pharmaceutically and physiologically acceptable formulation agents. Suitable pharmaceutically acceptable or physiologically acceptable diluents, carriers or excipients include, but are not limited to, antioxidants (e.g., ascorbic acid and sodium bisulfate), preservatives (e.g., benzyl alcohol, methyl parabens, ethyl or n-propyl, p-hydroxybenzoate), emulsifying agents, suspending agents, dispersing agents, solvents, fillers, bulking agents, detergents, buffers, vehicles, diluents, and/or adjuvants. For example, a suitable vehicle can be physiological saline solution or citrate buffered saline, possibly supplemented with other materials common in pharmaceutical compositions for parenteral administration. Neutral buffered saline or saline mixed with serum albumin are further exemplary vehicles. Those skilled in the art will readily recognize a variety of buffers that can be used in the pharmaceutical compositions and dosage forms contemplated herein. Typical buffers include, but are not limited to, pharmaceutically acceptable weak acids, weak bases, or mixtures thereof. As an example, the buffer components can be water soluble materials such as phosphoric acid, tartaric acids, lactic acid, succinic acid, citric acid, acetic acid, ascorbic acid, aspartic acid, glutamic acid, and salts thereof. Acceptable buffering agents include, for example, a Tris buffer, N-(2-Hydroxyethyl)piperazine-N′-(2-ethanesulfonic acid) (HEPES), 2-(N-Morpholino)ethanesulfonic acid (MES), 2-(N-Morpholino)ethanesulfonic acid sodium salt (MES), 3-(N-Morpholino)propanesulfonic acid (MOPS), and N-tris[Hydroxymethyl]methyl-3-aminopropanesulfonic acid (TAPS).

After a pharmaceutical composition has been formulated, it can be stored in sterile vials as a solution, suspension, gel, emulsion, solid, or dehydrated or lyophilized powder. Such formulations can be stored either in a ready-to-use form, a lyophilized form requiring reconstitution prior to use, a liquid form requiring dilution prior to use, or other acceptable form. In some embodiments, the pharmaceutical composition is provided in a single-use container (e.g., a single-use vial, ampoule, syringe, or autoinjector (similar to, e.g., an EpiPen®)), whereas a multi-use container (e.g., a multi-use vial) is provided in other embodiments. Any drug delivery apparatus can be used to deliver a PEG-IL-10 or an IL-12 agent, including implants (e.g., implantable pumps) and catheter systems, slow injection pumps and devices, all of which are well known to the skilled artisan. Depot injections, which are generally administered subcutaneously or intramuscularly, can also be utilized to release the polypeptides disclosed herein over a defined period of time. Depot injections are usually either solid- or oil-based and generally comprise at least one of the formulation components set forth herein. One of ordinary skill in the art is familiar with possible formulations and uses of depot injections.

The pharmaceutical compositions can be in the form of a sterile injectable aqueous or oleagenous suspension. This suspension can be formulated according to the known art using those suitable dispersing or wetting agents and suspending agents mentioned herein. The sterile injectable preparation can also be a sterile injectable solution or suspension in a non-toxic parenterally-acceptable diluent or solvent, for example, as a solution in 1,3-butane diol. Acceptable diluents, solvents and dispersion media that can be employed include water, Ringer's solution, isotonic sodium chloride solution, Cremophor EL™ (BASF, Parsippany, N.J.) or phosphate buffered saline (PBS), ethanol, polyol (e.g., glycerol, propylene glycol, and liquid polyethylene glycol), and suitable mixtures thereof. In addition, sterile, fixed oils are conventionally employed as a solvent or suspending medium. For this purpose, any bland fixed oil can be employed, including synthetic mono- or diglycerides. Moreover, fatty acids such as oleic acid, find use in the preparation of injectables. Prolonged absorption of particular injectable formulations can be achieved by including an agent that delays absorption (e.g., aluminum monostearate or gelatin).

The pharmaceutical compositions containing the active ingredient can be in a form suitable for oral use, for example, as tablets, capsules, troches, lozenges, aqueous or oily suspensions, dispersible powders or granules, emulsions, hard or soft capsules, or syrups, solutions, microbeads or elixirs. In particular embodiments, an active ingredient of an agent co-administered with a PEG-IL-10 and/or an IL-12 agent described herein is in a form suitable for oral use. Pharmaceutical compositions intended for oral use can be prepared according to any method known to the art for the manufacture of pharmaceutical compositions, and such compositions can contain one or more agents such as, for example, sweetening agents, flavoring agents, coloring agents and preserving agents in order to provide pharmaceutically elegant and palatable preparations. Tablets, capsules and the like contain the active ingredient in admixture with non-toxic pharmaceutically acceptable excipients which are suitable for the manufacture of tablets. These excipients can be, for example, diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc.

The tablets, capsules and the like suitable for oral administration can be uncoated or coated by known techniques to delay disintegration and absorption in the gastrointestinal tract and thereby provide a sustained action. For example, a time-delay material such as glyceryl monostearate or glyceryl distearate can be employed. They can also be coated by techniques known in the art to form osmotic therapeutic tablets for controlled release. Additional agents include biodegradable or biocompatible particles or a polymeric substance such as polyesters, polyamine acids, hydrogel, polyvinyl pyrrolidone, polyanhydrides, polyglycolic acid, ethylene-vinylacetate, methylcellulose, carboxymethylcellulose, protamine sulfate, or lactide/glycolide copolymers, polylactide/glycolide copolymers, or ethylenevinylacetate copolymers in order to control delivery of an administered composition. For example, the oral agent can be entrapped in microcapsules prepared by coacervation techniques or by interfacial polymerization, by the use of hydroxymethylcellulose or gelatin-microcapsules or poly (methylmethacrolate) microcapsules, respectively, or in a colloid drug delivery system. Colloidal dispersion systems include macromolecule complexes, nano-capsules, microspheres, microbeads, and lipid-based systems, including oil-in-water emulsions, micelles, mixed micelles, and liposomes. Methods for the preparation of the above-mentioned formulations will be apparent to those skilled in the art.

Formulations for oral use can also be presented as hard gelatin capsules wherein the active ingredient is mixed with an inert solid diluent, for example, calcium carbonate, calcium phosphate, kaolin or microcrystalline cellulose, or as soft gelatin capsules wherein the active ingredient is mixed with water or an oil medium, for example peanut oil, liquid paraffin, or olive oil.

Aqueous suspensions contain the active materials in admixture with excipients suitable for the manufacture thereof. Such excipients can be suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents, for example a naturally-occurring phosphatide (e.g., lecithin), or condensation products of an alkylene oxide with fatty acids (e.g., polyoxy-ethylene stearate), or condensation products of ethylene oxide with long chain aliphatic alcohols (e.g., for heptadecaethyleneoxycetanol), or condensation products of ethylene oxide with partial esters derived from fatty acids and a hexitol (e.g., polyoxyethylene sorbitol monooleate), or condensation products of ethylene oxide with partial esters derived from fatty acids and hexitol anhydrides (e.g., polyethylene sorbitan monooleate). The aqueous suspensions can also contain one or more preservatives.

Oily suspensions can be formulated by suspending the active ingredient in a vegetable oil, for example arachis oil, olive oil, sesame oil or coconut oil, or in a mineral oil such as liquid paraffin. The oily suspensions can contain a thickening agent, for example beeswax, hard paraffin or cetyl alcohol. Sweetening agents such as those set forth above, and flavoring agents can be added to provide a palatable oral preparation.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified herein.

The pharmaceutical compositions of the present disclosure can also be in the form of oil-in-water emulsions. The oily phase can be a vegetable oil, for example olive oil or arachis oil, or a mineral oil, for example, liquid paraffin, or mixtures of these. Suitable emulsifying agents can be naturally occurring gums, for example, gum acacia or gum tragacanth; naturally occurring phosphatides, for example, soy bean, lecithin, and esters or partial esters derived from fatty acids; hexitol anhydrides, for example, sorbitan monooleate; and condensation products of partial esters with ethylene oxide, for example, polyoxyethylene sorbitan monooleate.

Formulations can also include carriers to protect the composition against rapid degradation or elimination from the body, such as a controlled release formulation, including implants, liposomes, hydrogels, prodrugs and microencapsulated delivery systems. For example, a time delay material such as glyceryl monostearate or glyceryl stearate alone, or in combination with a wax, can be employed.

The present disclosure contemplates the administration of the IL-10 polypeptides in the form of suppositories for rectal administration. The suppositories can be prepared by mixing the drug with a suitable non-irritating excipient which is solid at ordinary temperatures but liquid at the rectal temperature and will therefore melt in the rectum to release the drug. Such materials include, but are not limited to, cocoa butter and polyethylene glycols.

The PEG-IL-10 and IL-12 agents contemplated by the present disclosure can be in the form of any other suitable pharmaceutical composition (e.g., sprays for nasal or inhalation use) currently known or developed in the future.

The concentration of a polypeptide or fragment thereof in a formulation can vary widely (e.g., from less than about 0.1%, usually at or at least about 2% to as much as 20% to 50% or more by weight) and will usually be selected primarily based on fluid volumes, viscosities, and subject-based factors in accordance with, for example, the particular mode of administration selected.

### Routes of Administration

The present disclosure contemplates the administration of the PEG-IL-10 and IL-12 agents, and compositions thereof, in any appropriate manner. Suitable routes of administration include parenteral (e.g., intramuscular, intravenous, subcutaneous (e.g., injection or implant), intraperitoneal, intracisternal, intraarticular, intraperitoneal, intracerebral (intraparenchymal) and intracerebroventricular), oral, nasal, vaginal, sublingual, intraocular, rectal, topical (e.g., transdermal), sublingual and inhalation. Depot injections, which are generally administered subcutaneously or intramuscularly, can also be utilized to release the polypeptides disclosed herein over a defined period of time.

Particular embodiments of the present disclosure contemplate parenteral administration. The parenteral administration is intravenous in some embodiments and is subcutaneous in others.

### Supplementary Combination Therapy

The present disclosure contemplates the use of the combinations of PEG-IL-10 and an IL-12 agent in further combination with one or more active therapeutic agents or other prophylactic or therapeutic modalities (e.g., radiation). For purposes of this application, such further combinations are sometimes referred to as “supplementary combinations”, “supplementary combination therapy”, “combinations with an additional prophylactic or therapeutic agent” and the like, and agents that are added to combinations of PEG-IL-10 and an IL-12 agent can be referred to as “supplementary agents” and the like. In such supplementary combination therapy, the various supplementary active agent(s) frequently have different mechanisms of action than a PEG-IL-10 and/or an IL-12 agent. Such supplementary combination therapy can be especially advantageous by allowing a dose reduction of one or more of the agents, thereby reducing or eliminating the adverse effects associated with one or more of the agents; furthermore, such supplementary combination therapy can have a synergistic therapeutic or prophylactic effect on the underlying proliferative disease, disorder, or condition. In some embodiments of the present disclosure the supplementary agent(s) is a diagnostic agent(s).

In particular embodiments, the present disclosure provides methods for treating and/or preventing cancer-related diseases, disorders or conditions with a PEG-IL-10 and an IL-12 agent, and at least one additional therapeutic or diagnostic agent.

In some embodiments of the present disclosure, each of the PEG-IL-10, the IL-12 agent and the supplementary agent(s) can be in a separate dosage form. By way of example, the PEG-IL-10 can be in a formulation suitable for SC administration, the IL-12 agent can be in a formulation suitable for IV administration, and the supplementary agent can be in a formulation suitable for oral administration; in this context, each of the agents can be housed separately or two or more of the agents can be housed together (e.g., as distinct components of a kit). In other embodiments of the present disclosure, two or more of the PEG-IL-10, the IL-12 agent and the supplementary agent(s) are in the same dosage form. For example, the PEG-IL-10, the IL-12 agent, and the supplementary agent(s) can be formulated for IV administration; in this context, one or more of the agents can be co-formulated (e.g., as the active therapeutic agents in a syringe).

In certain embodiments, the PEG-IL-10, the IL-12 agent, and the supplemental agent(s) (e.g., a chemotherapeutic agent) are administered or applied sequentially, e.g., where the PEG-IL-10 is administered first, an IL-12 agent is administered second, and a supplemental agent is administered last. In other embodiments, the PEG-IL-10, the IL-12 agent, and the supplemental agent(s) are administered simultaneously, e.g., where two of them are administered simultaneously and the third is administered either before or after. Regardless of whether the PEG-IL-10, the IL-12 agent, and the supplemental agent(s) are administered sequentially, simultaneously, or some variation thereof, they are considered to be administered as supplementary combination therapy for purposes of the present disclosure.

The present disclosure contemplates the use of any possible dosing regimen for the supplementary combination therapy that may be acceptable, appropriate or optimal under the circumstances. The regimens described hereafter are exemplary, not exclusionary. In one embodiment, treatment with the PEG-IL-10, an IL-12 agent, and the supplemental agent(s) are maintained over a period of time. In another embodiment, treatment with the PEG-IL-10, an IL-12 agent, and the supplemental agent(s) are reduced or continued over a period to time (e.g., when the subject is stable). In another embodiment, treatment with the supplemental agent(s) is reduced or discontinued (e.g., when the subject is stable), while treatment with the PEG-IL-10 and an IL-12 agent is maintained at a constant dosing regimen. In a further embodiment, treatment with the supplemental agent(s) is reduced or discontinued (e.g., when the subject is stable), treatment with the PEG-IL-10 is reduced (e.g., lower dose, less frequent dosing or shorter treatment regimen), and treatment with the IL-12 agent is maintained at a constant dosing regimen. In a further embodiment, treatment with the supplemental agent(s) is reduced or discontinued (e.g., when the subject is stable), treatment with the PEG-IL-10 is reduced (e.g., lower dose, less frequent dosing or shorter treatment regimen), and treatment with IL-12 agent is maintained at a constant dosing regimen.

In yet another embodiment, treatment with the supplemental agent(s) and the PEG-IL-10 is maintained at a constant dosing regimen, while treatment with the IL-12 agent is reduced or discontinued (e.g., when the subject is stable). In yet a further embodiment, treatment with the supplemental agent(s) and the IL-12 agent is maintained at a constant dosing regimen, while treatment with the PEG-IL-10 is reduced or discontinued (e.g., lower dose, less frequent dosing or shorter treatment regimen). Identification and use of other dosing regimens will be apparent to the skilled artisan.

While particular agents suitable for use with the combinations of a PEG-IL-10 and an IL-12 agent disclosed herein are set forth hereafter, it is to be understood that the present disclosure is not so limited. By way of example, but not limitation, a prophylactic or therapeutic agent may be a chemotherapeutic agent, an immune- or inflammation-related agent, a metabolic agent, an antiviral agent or an anti-thrombotic agent. The methods of the present disclosure may also be used in combination with non-pharmacological agents (e.g., radiology).

In a particular embodiment, the present disclosure contemplates the use of a PEG-IL-10 and an IL-12 agent with a chemotherapeutic agent(s) for treating and/or preventing cancer, tumor, or precancerous or cancer-associated disease, disorder or condition. Examples of chemotherapeutic agents include, but are not limited to, alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chiorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); cyclophosphamide; thiotepa; taxoids, e.g., paclitaxel and doxetaxel; chlorambucil; gemcitabine; 6-thioguanine; mercaptopurine; methotrexate; platinum and platinum coordination complexes such as cisplatin and carboplatin; vinblastine; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitors; difluoromethylornithine (DMFO); retinoic acid; esperamicins; capecitabine; and pharmaceutically acceptable salts, acids or derivatives of any of the above.

Chemotherapeutic agents also include anti-hormonal agents that act to regulate or inhibit hormone action on tumors such as anti-estrogens, including for example tamoxifen, raloxifene, aromatase inhibiting 4(5)-imidazoles, 4-hydroxytamoxifen, trioxifene, keoxifene, onapristone, and toremifene; and antiandrogens such as flutamide, nilutamide, bicalutamide, leuprolide, and goserelin; and pharmaceutically acceptable salts, acids or derivatives of any of the above. In certain embodiments, combination therapy comprises administration of a hormone or related hormonal agent.

Any other agent useful in the treatment or prevention of the cancerous conditions described herein is contemplated as a supplementary agent, including, but not limited to, a cytokine or cytokine antagonist, such as IL-12, INFα, or anti-epidermal growth factor receptor, radiotherapy, a monoclonal antibody against another tumor antigen, a complex of a monoclonal antibody and toxin, a T-cell adjuvant, bone marrow transplant, or antigen presenting cells (e.g., dendritic cell therapy). Vaccines (e.g., as a soluble protein or as a nucleic acid encoding the protein) are also provided herein.

In particular embodiments, the additional prophylactic or therapeutic agent is a chemotherapeutic agent, examples of which are set forth herein. In some embodiments, the chemotherapeutic agent is a platinum-based antineoplastic, also referred to as a platinum coordination complex. These platinum-based antineoplastic agents crosslink DNA, thereby inhibiting DNA repair and/or DNA synthesis in cancer cells. Examples of such agents include cisplatin, carboplatin, oxaliplatin, satraplatin, picoplatin, nedaplatin and triplatin

The present disclosure encompasses pharmaceutically acceptable salts, acids or derivatives of any of the above.

### Dosing

A PEG-IL-10 and an IL-12 agent of the present disclosure can be administered to a subject in an amount that is dependent upon, for example, the goal of the administration (e.g., the degree of resolution desired); the age, weight, sex, and health and physical condition of the subject the formulation being administered; the route of administration; and the nature of the disease, disorder, condition or symptom thereof. The dosing regimen can also take into consideration the existence, nature, and extent of any adverse effects associated with the agent(s) being administered. Effective dosage amounts and dosage regimens can readily be determined from, for example, safety and dose-escalation trials, in vivo studies (e.g., animal models), and other methods known to the skilled artisan.

As discussed elsewhere herein, the present disclosure contemplates embodiments of the PEG-IL-10 and IL-12 agent combination therapy wherein a PEG-IL-10 is administered in an amount and frequency such that a desired serum trough concentration (e.g., ≥10 ng/mL) is maintained. Embodiments of the of the PEG-IL-10 and IL-12 agent combination therapy are also contemplated wherein an IL-12 agent is dosed such that the serum concentration achieves a peak and is then cleared to an unmeasurable level before it is administered again.

In general, dosing parameters dictate that the dosage amount be less than an amount that could be irreversibly toxic to the subject (i.e., the maximum tolerated dose, “MTD”) and not less than an amount required to produce a measurable effect on the subject. Such amounts are determined by, for example, the pharmacokinetic and pharmacodynamic parameters associated with ADME, taking into consideration the route of administration and other factors.

As used herein, the term “EC50” and the phrase “half maximal effective concentration” have their generally accepted meaning; that is, the EC50 is the concentration of a therapeutic agent (e.g., a PEG-IL-10) which induces a response halfway between the baseline and the maximum after some specified exposure time. The skilled artisan is familiar with means for determining the EC50 of a therapeutic agent. For example, the EC50 may be determined using commercially available software (e.g., Graphpad Software, Inc.; La Jolla, Calif.) after measuring certain concentration-related parameters of the therapeutic agent in a cell-based assay.

An effective dose (ED) is the dose or amount of an agent that produces a therapeutic response or desired effect in some fraction of the subjects taking it. The “median effective dose” or ED50 of an agent is the dose or amount of an agent that produces a therapeutic response or desired effect in 50% of the population to which it is administered. Although the ED50 is commonly used as a measure of reasonable expectance of an agent's effect, it is not necessarily the dose that a clinician might deem appropriate taking into consideration all relevant factors. Thus, in some situations the effective amount can be more than the calculated ED50, in other situations the effective amount can be less than the calculated ED50, and in still other situations the effective amount can be the same as the calculated ED50.

In addition, an effective dose of a PEG-IL-10 and an IL-12 agent of the present disclosure can be an amount that, when administered in one or more doses to a subject, produces a desired result relative to a healthy subject. For example, for a subject experiencing a particular disorder, an effective dose can be one that improves a diagnostic parameter, measure, marker and the like of that disorder by at least about 5%, at least about 10%, at least about 20%, at least about 25%, at least about 30%, at least about 40%, at least about 50%, at least about 60%, at least about 70%, at least about 80%, at least about 90%, or more than 90%, where 100% is defined as the diagnostic parameter, measure, marker and the like exhibited by a normal subject.

The amount of a PEG-IL-10 and an IL-12 agent necessary to treat a disease, disorder or condition described herein can be determined by activity assays known in the art. By way of example, in the tumor context, suitable IL-10 activity includes, for example, CD8+ T-cell infiltrate into tumor sites, expression of inflammatory cytokines, such as IFN-γ, IL-4, IL-6, IL-10, and RANK-L, from these infiltrating cells, and increased levels of TNFα or IFNγ in biological samples.

The therapeutically effective amount of PEG-IL-10 can range from about 0.01 to about 100 μg protein/kg of body weight/day, from about 0.1 to 20 μg protein/kg of body weight/day, from about 0.5 to 10 μg protein/kg of body weight/day, or about 1 to 4 μg protein/kg of body weight/day. The present disclosure contemplates embodiments wherein the amount of the PEG-IL-10 component of the combination therapy is from 10.0 μg/kg/day to 20.0 μg/kg/day. In some embodiments, the amount of the PEG-IL-10 administered is from 12.0 μg/kg/day to 18.0 μg/kg/day.

In some embodiments, PEG-IL-10 component of the combination therapy is administered (e.g., by continuous infusion) so as to provide for delivery of about 50 to 800 μg protein/kg of body weight/day (e.g., about 1 to 16 μg protein/kg of body weight/day of PEG-IL-10). Where delivered by infusion, the infusion rate can be varied based on evaluation of, for example, adverse effects and blood cell counts. Other specific dosing parameters for a PEG-IL-10 are described elsewhere herein.

The present disclosure contemplates embodiments wherein the amount of the IL-12 component of the PEG-IL-10 combination therapy that is administered to the subject to treat or prevent a cancer-related disease, disorder or condition is from 0.01 μg/kg/day to 10.0 μg/kg/day. In other embodiments, the amount of the IL-12 agent is from 0.1 μg/kg/day to 10.0 μg/kg/day, and in still other embodiments the amount of the IL-12 agent is from 1.0 μg/kg/day to 10.0 μg/kg/day. In still further embodiments, the amount of the IL-12 component of the combination therapy that is administered to the subject to treat or prevent a cancer-related disease, disorder or condition is from 0.1 μg/kg/day to 15.0 μg/kg/day. In other embodiments, the amount of the IL-12 agent is from 1.0 μg/kg/day to 15.0 μg/kg/day, and in still other embodiments the amount of the IL-12 agent is from 10.0 μg/kg/day to 15.0 μg/kg/day.

The present disclosure contemplates embodiments of the PEG-IL-10/IL-12 agent combination therapy in which the amount of PEG-IL-10 administered is 10.0 μg/kg/day to 20.0 μg/kg/day; 11.0 μg/kg/day to 19.0 μg/kg/day; 12.0 μg/kg/day to 18.0 μg/kg/day; 13.0 μg/kg/day to 17.0 μg/kg/day; 14.0 μg/kg/day to 16.0 μg/kg/day; or about 15.0 μg/kg/day. The present disclosure contemplates embodiments of the PEG-IL-10/IL-12 agent combination therapy in which the amount of IL-12 agent administered is 0.01 μg/kg/day to 10.0 μg/kg/day; from 0.05 μg/kg/day to 9.5 μg/kg/day; 0.1 μg/kg/day to 10.0 μg/kg/day; 0.1 μg/kg/day to 9.0 μg/kg/day; 0.5 μg/kg/day to 8.5 μg/kg/day; 1.0 μg/kg/day to 10.0 μg/kg/day; 1.0 μg/kg/day to 8.0 μg/kg/day; 1.5 μg/kg/day to 7.5 μg/kg/day; 2.0 μg/kg/day to 7.0 μg/kg/day; 2.5 μg/kg/day to 6.5 μg/kg/day; 3.0 μg/kg/day to 6.0 μg/kg/day; 3.5 μg/kg/day to 5.5 μg/kg/day; 4.0 μg/kg/day to 5.0 μg/kg/day; or 4.5 μg/kg/day, which may be administered in combination with any of the amounts of PEG-IL-10 set out herein (e.g., PEG-IL-10 administered in an amount of 10.0 μg/kg/day to 20.0 μg/kg/day; 11.0 μg/kg/day to 19.0 μg/kg/day; 12.0 μg/kg/day to 18.0 μg/kg/day; 13.0 μg/kg/day to 17.0 μg/kg/day; 14.0 μg/kg/day to 16.0 μg/kg/day; or about 15.0 μg/kg/day).

For administration of an oral agent, the compositions can be provided in the form of tablets, capsules and the like containing from 1.0 to 1000 milligrams of the active ingredient, particularly 1.0, 3.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient.

In certain embodiments, the dosage of the disclosed PEG-IL-10 and/or IL-12 agent is contained in a “unit dosage form”. The phrase “unit dosage form” refers to physically discrete units, each unit containing a predetermined amount of a PEG-IL-10 and/or an IL-12 agent of the present disclosure, either alone or in combination with one or more additional agents, sufficient to produce the desired effect. It will be appreciated that the parameters of a unit dosage form will depend on the particular agent and the effect to be achieved.

### Kits

The present disclosure also contemplates kits comprising PEG-IL-10 and/or an IL-12 agent, and pharmaceutical compositions thereof. The kits are generally in the form of a physical structure housing various components, as described below, and can be utilized, for example, in practicing the methods described above. One or more components of a kit can be in a sterile container (e.g., a sterile vial).

A kit can include a PEG-IL-10 and/or an IL-12 agent disclosed herein, which can be in the form of a pharmaceutical composition suitable for administration to a subject. The PEG-IL-10 and/or IL-12 agent can be provided in a form that is ready for use or in a form requiring, for example, reconstitution or dilution prior to administration. When the PEG-IL-10 and/or IL-12 agent is in a form that needs to be reconstituted by a user, the kit can also include buffers, pharmaceutically acceptable excipients, and the like, packaged with or separately from the PEG-IL-10 and/or IL-12 agent. A kit can also contain both the PEG-IL-10 and an IL-12 agent as described herein; the kit can contain the several agents separately or they can already be combined in the kit. Similarly, when supplementary therapy (e.g., a PEG-IL-10, an IL-12 agent, and a supplementary agent) is contemplated, the kit can contain the several agents separately or two or more of them can already be combined in the kit. A kit of the present disclosure can be designed for conditions necessary to properly maintain the components housed therein (e.g., refrigeration or freezing).

A kit can contain a label or packaging insert including identifying information for the components therein and instructions for their use (e.g., dosing parameters, clinical pharmacology of the active ingredient(s), including mechanism(s) of action, pharmacokinetics and pharmacodynamics, adverse effects, contraindications, etc.). Each component of the kit can be enclosed within an individual container, and all of the various containers can be within a single package. Labels or inserts can include manufacturer information such as lot numbers and expiration dates. The label or packaging insert can be, e.g., integrated into the physical structure housing the components, contained separately within the physical structure, or affixed to a component of the kit (e.g., an ampule, syringe or vial).

Labels or inserts can additionally include, or be incorporated into, a computer readable medium, such as a disk (e.g., hard disk, card, memory disk), optical disk such as CD- or DVD-ROM/RAM, DVD, MP3, magnetic tape, or an electrical storage media such as RAM and ROM or hybrids of these such as magnetic/optical storage media, FLASH media or memory-type cards. In some embodiments, the actual instructions are not present in the kit, but means for obtaining the instructions from a remote source, e.g., via an internet site, are provided.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that can be performed. It is to be understood that exemplary descriptions written in the present tense were not necessarily performed, but rather that the descriptions can be performed to generate the data and the like described therein. Efforts have been made to ensure accuracy with respect to numbers used (e.g., amounts, temperature, etc.), but some experimental errors and deviations should be accounted for.

Unless indicated otherwise, parts are parts by weight, molecular weight is weight average molecular weight, temperature is in degrees Celsius (° C.), and pressure is at or near atmospheric. Standard abbreviations are used, including the following: s or sec=second(s); min=minute(s); h or hr=hour(s); aa=amino acid(s); bp=base pair(s); kb=kilobase(s); nt=nucleotide(s); ng=nanogram; μg=microgram; mg=milligram; g=gram; kg=kilogram; dl or dL=deciliter; μl or μL=microliter; ml or mL=milliliter; 1 or L=liter; nM=nanomolar; μM=micromolar; mM=millimolar; M=molar; kDa=kilodalton; i.m.=intramuscular(ly); i.p.=intraperitoneal(ly); SC or SQ=subcutaneous(ly); HPLC=high performance liquid chromatography; BW=body weight; U=unit; ns=not statistically significant; PMA=Phorbol 12-myristate 13-acetate; PBS=phosphate-buffered saline; HSA=human serum albumin; DMEM=Dulbeco's Modification of Eagle's Medium; PBMCs=primary peripheral blood mononuclear cells; FBS=fetal bovine serum; FCS=fetal calf serum; HEPES=4-(2-hydroxyethyl)-1-piperazineethanesulfonic acid; LPS=lipopolysaccharide; ATCC=American Type Culture Collection.

### Materials and Methods.

The following general materials and methods were used, where indicated, or can be used in the Examples below:

Molecular Biology Procedures. Standard methods in molecular biology are described in the scientific literature (see, e.g., Sambrook and Russell (2001) Molecular Cloning, 3^{rd} ed., Cold Spring Harbor Laboratory Press, Cold Spring Harbor, N.Y.; and Ausubel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4)).

Antibody-related Processes. Production, purification, and fragmentation of polyclonal and monoclonal antibodies are described (e.g., Harlow and Lane (1999) Using Antibodies, Cold Spring Harbor Laboratory Press, Cold Spring Harbor, NY); standard techniques for characterizing ligand/receptor interactions are available (see, e.g., Coligan et al. (2001) Current Protocols in Immunology, Vol. 4, John Wiley, Inc., NY); methods for flow cytometry, including fluorescence-activated cell sorting (FACS), are available (see, e.g., Shapiro (2003) Practical Flow Cytometry, John Wiley and Sons, Hoboken, N.J.); and fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.). Further discussion of antibodies appears elsewhere herein.

Software. Software packages and databases for determining, e.g., antigenic fragments, leader sequences, protein folding, functional domains, glycosylation sites, and sequence alignments, are available (see, e.g., GCG Wisconsin Package (Accelrys, Inc., San Diego, Calif.); and DeCypher™ (TimeLogic Corp., Crystal Bay, Nev.).

Pegylation. Pegylated IL-10 as described herein can be synthesized by any means known to the skilled artisan. Exemplary synthetic schemes for producing mono-PEG-IL-10 and a mix of mono-/di-PEG-IL-10 have been described (see, e.g., U.S. Pat. No. 7,052,686; US Pat. Publn. No. 2011/0250163; WO 2010/077853). Particular embodiments of the present disclosure comprise a mix of selectively pegylated mono- and di-PEG-IL-10. In addition to leveraging her own skills in the production and use of PEGs (and other drug delivery technologies) suitable in the practice of the present disclosure, the skilled artisan is familiar with many commercial suppliers of PEG-related technologies (e.g., NO America Corp (Irvine, Calif.) and Parchem (New Rochelle, N.Y.)).

Mice. Various mice and other animal strains can be used in conjunction with the teachings of the present disclosure. For example, immunocompetent Balb/C or B-cell—deficient Balb/C mice can be obtained from The Jackson Lab., Bar Harbor, Me. and used in accordance with standard procedures (see, e.g., Martin et al (2001) Infect. Immun., 69(11):7067-73 and Compton et al. (2004) Comp. Med. 54(6):681-89). Other mice strains suitable for the experimental work contemplated by the present disclosure are known to the skilled artisan and are generally available from The Jackson Lab or another supplier.

IL-10 Concentrations. Serum IL-10 concentration levels and exposure levels can be determined by standard methods used in the art. For example, a serum exposure level assay can be performed by collecting whole blood (˜50 μL/mouse) from mouse tail snips into plain capillary tubes, separating serum and blood cells by centrifugation, and determining IL-10 exposure levels by standard ELISA kits and techniques.

The assays described hereafter are representative, and not exclusionary.

In Vitro Cytokine Secretion Assay. Activated primary human CD8+ T-cells secrete IFN-γ when treated with PEG-IL-10 and then with an anti-CD3 antibody. The following protocol provides an exemplary assay to examine cytokine secretion.

Human PBMCs can be isolated according to any standard protocol (see, e.g., Fuss et al. (2009) Current Protocols in Immunology, Unit 7.1, John Wiley, Inc., NY). CD8+ T-cells can be isolated using Miltenyi Biotec's MACS cell separation technology according to the manufacture's protocol (Miltenyi Biotec; Auburn, Calif.). For assays during activation, the isolated CD8+ T-cells (2×10⁶ cells/mL, 5×10⁵ cells per well of a standard 96-well plate) can be activated with plate-bound anti-CD3 and anti-CD28 (plates are pre-coated with 10 μg/mL anti-CD3 and 2 μg/mL anti-CD28; Affymetrix eBioscience; San Diego, Calif.) and appropriate concentrations of IL-12 or PEG-IL-10 for 3 days in AIM V media (Life Technologies; Carlsbad, Calif.). The media can then be collected and assayed for IFN-γ using a commercially available ELISA kit following the manufacture's protocol (Affymetrix eBioscience; San Diego, Calif.). For assays during the rest phase, the isolated CD8+ T-cells (3×10⁶ cells/mL, 3×10⁶ cells per well of a standard 24-well plate) can be activated with plate-bound anti-CD3 and anti-CD28 (plates are pre-coated with 10 μg/mL anti-CD3 and 2 μg/mL anti-CD28; Affymetrix eBioscience; San Diego, Calif.) for 3 days. Following activation, cells can then be collected, re-plated (2×10⁶ cells/mL, 5×10⁵ cells per well of a standard 96-well plate) and treated with appropriate concentrations of IL-12 or PEG-hIL-10 for 3 days in AIM V media. After treatment, cells can be collected, re-plated (2×0⁶ cells/mL, 5×10⁵ cells per well of a standard 96-well plate) and treated with 1 μg/mL soluble anti-CD3 for 4 hrs in AIM V media. The media can then be collected and assayed for IFN-γ (Affymetrix eBioscience; San Diego, Calif.), Granzyme B and Perforin (Mabtech; Cincinnati, Ohio) using commercially available ELISA kits following the manufacture's protocol.

TNFα Inhibition Assay. PMA-stimulation of U937 cells (lymphoblast human cell line from lung available from Sigma-Aldrich (#85011440); St. Louis, Mo.) causes the cells to secrete TNFα, and subsequent treatment of these TNFα—secreting cells with human IL-10 causes a decrease in TNFα secretion in a dose-dependent manner. An exemplary TNFα inhibition assay can be performed using the following protocol.

After culturing U937 cells in RMPI containing 10% FBS/FCS and antibiotics, plate 1×105, 90% viable U937 cells in 96-well flat bottom plates (any plasma-treated tissue culture plates (e.g., Nunc; Thermo Scientific, USA) can be used) in triplicate per condition. Plate cells to provide for the following conditions (all in at least triplicate; for ‘media alone’ the number of wells is doubled because one-half will be used for viability after incubation with 10 nM PMA): 5 ng/mL LPS alone; 5 ng/mL LPS +0.1 ng/mL rhIL-10; 5 ng/mL LPS +1 ng/mL rhIL-10; 5 ng/mL LPS +10 ng/mL rhIL-10; 5 ng/mL LPS +100 ng/mL rhIL-10; 5 ng/mL LPS +1000 ng/mL rhlL-10; 5 ng/mL LPS +0.1 ng/mL PEG-rhIL-10; 5 ng/mL LPS +1 ng/mL PEG-rhIL-10; 5 ng/mL LPS +10 ng/mL PEG-rhIL-10; 5 ng/mL LPS +100 ng/mL PEG-rhIL-10; and 5 ng/mL LPS +1000 ng/mL PEG-rhIL-10. Expose each well to 10 nM PMA in 200 μL for 24 hours, culturing at 37° C. in 5% CO₂ incubator, after which time ˜90% of cells should be adherent. The three extra wells can be re-suspended, and the cells are counted to assess viability (>90% should be viable). Wash gently but thoroughly 3× with fresh, non-PMA—containing media, ensuring that cells are still in the wells. Add 100 μL per well of media containing the appropriate concentrations (2× as the volume will be diluted by 100%) of rhIL-10 or PEG-rhIL-10, incubate at 37° C. in a 5% CO₂ incubator for 30 minutes. Add 100 μL per well of 10 ng/mL stock LPS to achieve a final concentration of 5 ng/mL LPS in each well, and incubate at 37° C. in a 5% CO₂ incubator for 18-24 hours. Remove supernatant and perform TNFα ELISA according to the manufacturer's instructions. Run each conditioned supernatant in duplicate in ELISA.

MC/9 Cell Proliferation Assay. IL-10 administration to MC/9 cells (murine cell line with characteristics of mast cells available from Cell Signaling Technology; Danvers, Mass.) causes increased cell proliferation in a dose-dependent manner. Thompson-Snipes, L. et al. (1991) J. Exp. Med. 173:507-10) describe a standard assay protocol in which MC/9 cells are supplemented with IL3+IL-10 and IL-3+IL-4+ IL-10. Vendors (e.g., R&D Systems, USA; and Cell Signaling Technology, Danvers, Mass.) use the assay as a lot release assay for rhIL-10. Those of ordinary skill in the art will be able to modify the standard assay protocol described in Thompson-Snipes, L. et al, such that cells are only supplemented with IL-10.

Tumor Models and Tumor Analysis. Any art-accepted tumor model, assay, and the like can be used to evaluate the effect of the IL-10 molecules described herein on various tumors. The tumor models and tumor analyses described hereafter are representative of those that can be utilized. Syngeneic mouse tumor cells are injected subcutaneously or intradermally at 10⁴, 10⁵ or 10⁶ cells per tumor inoculation. Ep2 mammary carcinoma, CT26 colon carcinoma, PDV6 squamous carcinoma of the skin and 4T1 breast carcinoma models can be used (see, e.g., Langowski et al. (2006) Nature 442:461-465). Immunocompetent Balb/C or B-cell deficient Balb/C mice can be used. PEG 10-mIL-10 can be administered to the immunocompetent mice, while PEG-hIL-10 treatment can be in the B-cell deficient mice. Tumors are allowed to reach a size of 100-250 mm³ before treatment is started. IL-10, PEG-mIL-10, PEG-hIL-10, or buffer control is administered SC at a site distant from the tumor implantation. Tumor growth is typically monitored twice weekly using electronic calipers. Tumor tissues and lymphatic organs are harvested at various endpoints to measure mRNA expression for a number of inflammatory markers and to perform immunohistochemistry for several inflammatory cell markers. The tissues are snap-frozen in liquid nitrogen and stored at −80° C. Primary tumor growth is typically monitored twice weekly using electronic calipers. Tumor volume can be calculated using the formula (width²×length/2) where length is the longer dimension. Tumors are allowed to reach a size of 90-250 mm³ before treatment is started.

### EXAMPLE 1

### Anti-Tumor Effect of PEG-IL-10 in Combination with IL-12

This example demonstrates the combinatorial effect of PEG-IL-10 and IL-12 on tumor size in a murine 4T1 tumor model.

Briefly, 1×10⁴ 4T1 cells (CRL-2539; ATCC, Manassas, Va.) in a volume of 100 μl were implanted SC into the right lower flank of female BALB/c mice (Jackson Laboratory, Bar Harbor, Me.) of 4-6 weeks of age. Once palpable, tumor growth was measured twice weekly—tumor volume can be calculated using the formula (width²×length/2), where length is the longer dimension. When tumors reached an average of 75 mm³ in volume, animals were stratified.

Eight mice per cohort were administered vehicle, and/or 1 mg/kg PEG-rMuIL-10 (ARMO Biosciences, Redwood City, Calif.), and/or 0.05, 0.1, or 0.5 mg/kg rMuIL-12 (R&D Systems, Minneapolis, Minn.) SC daily for 21 or 28 days. Each mouse received two separate injections (e.g., IL-10 and vehicle, or IL-10 and IL-12, or vehicle and vehicle). After 21 days of dosing, 4 mice from each group were sacrificed for tissue and tumor analysis. After 28 days of dosing, the remaining mice from each group were sacrificed for tissue and tumor analysis.

Tumor weights were assessed after 21 days, and the data are presented in FIG. 2. The amount of rMuIL-12 adminsitered is indicated on the X-axis; as noted above where PEG-rMuIL-10 was administered, the dose was 1 mg/kg. As indicated in FIG. 2, administration of each of the combinations of PEG-rMuIL-10 and rMuIL-12 resulted in a larger reduction in tumor weight than the administration of either agent alone. This effect was more pronounced at the higher doses of rMuIL-12 (i.e., 0.5 and 0.1 mg/kg; *=P<0.05). The bars in FIG. 2 represent the mean of the individual mouse data. Mice evaluated after 28 days exhibited the same general trends (data not shown).

### EXAMPLE 2

### Effect of PEG-IL-10 in Combination with IL-12 on Serum Cytokine Levels

This example demonstrates the combinatorial effect of PEG-IL-10 and IL-12 on serum IFNγ and TNFα levels in tumor-bearing mice. As described herein, exposure to each of IL-10 and IL-12 individually, and particularly IL-12, leads to the induction of the serum cytokines IFNγ and TNFα. The increased serum levels of IFNγ and TNFα (though primarily IFNγ) are associated with IL-12's systemic toxicity.

Briefly, IFNγ and TNFα levels were evaluated in the mice described in Example 1 (i.e., mice administered vehicle, 1 mg/kg PEG-rMuIL-10, and/or 0.05, 0.1, or 0.5 mg/kg rMuIL-12 SC daily) after 9 days of dosing, 4 hours after dose administration. Plasma cytokine levels were detected using Meso Scale Discovery's V-PLEX Proinflammatory Panell (mouse) Kit (Rockville, Md.), performed according to the manufacturer's instructions. The results are provided in FIG. 3A and FIG. 3B. The amount of rMuIL-12 adminsitered is indicated on the X-axis in each of FIGS. 3A and 3B; as noted above, where PEG-rMuIL-10 was administered, the dose was 1 mg/kg.

As indicated in FIG. 3A, co-administration of PEG-rMuIL-10 with each of the three rMuIL-12 doses resulted in decreases in the serum IFNγ levels observed following administration of each of the three rMuIL-12 doses alone. Moreover, when 0.5 mg/kg rMuIL-12 was co-administered with 1 mg/kg PEG-rMuIL-10, there was a statistically signification decrease (***=P<0.001) in the serum IFNγ levels as compared to administration of 0.5 mg/kg rMuIL-12 alone. These data are representative of the effect that PEG-IL-10 has when co-administered with IL-12—the enhanced anti-tumor response resulting from combination therapy (see FIG. 2) is not compromised while the putative toxicity “index” associated with IL-12 is reduced.

As indicated in FIG. 3B, co-administration of PEG-rMuIL-10 with each of the three rMuIL-12 doses resulted in decreases in the serum TNFα levels observed following administration of each of the three rMuIL-12 doses alone. Moreover, when 0.5 mg/kg rMuIL-12 was co-administered with 1 mg/kg PEG-rMuIL-10, there was a statistically signification decrease (***=P<0.001) in the serum TNFα levels as compared to administration of 0.5 mg/kg rMuIL-12 alone. These data are representative of the effect that PEG-IL-10 has when co-administered with IL-12—the enhanced anti-tumor response resulting from combination therapy (see FIG. 2) is not compromised while the putative toxicity “index” associated with IL-12 is reduced.

Particular embodiments of this invention are described herein, including the best mode known to the inventors for carrying out the invention. Upon reading the foregoing, description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

All publications, patent applications, accession numbers, and other references cited in this specification are herein incorporated by reference as if each individual publication or patent application were specifically and individually indicated to be incorporated by reference.

1. A method of treating or preventing a cancer-related disease, disorder or condition in a subject, comprising administering to the subject:
a) a therapeutically effective amount of an IL-12 agent, and
b) a therapeutically effective amount of a PEG-IL-10;
wherein the amount of the PEG-IL-10 is sufficient to reduce the IL-12—associated toxicity to a level less than that observed with IL-12 monotherapy.

2. A method of treating or preventing a cancer-related disease, disorder or condition in a subject, comprising administering to the subject:
a) a therapeutically effective amount of an IL-12 agent; and
b) a therapeutically effective amount of a PEG-IL-10, wherein the amount is sufficient to i) achieve a mean IL-10 serum trough concentration of at least 1.0 ng/mL, and ii) reduce the IL-12—associated toxicity to a level less than that observed with IL-12 monotherapy.

3. A method of treating or preventing a cancer-related disease, disorder or condition in a subject, comprising administering to the subject:
a) a therapeutically effective amount of an IL-12 agent; and
b) a therapeutically effective amount of a PEG-IL-10, wherein the amount is sufficient to:
i) maintain a mean IL-10 serum trough concentration over a period of time, wherein the mean IL-10 serum trough concentration is at least 1.0 ng/mL, and wherein the mean IL-10 serum trough concentration is maintained for at least 90% of the period of time; and
ii) reduce the IL-12—associated toxicity to a level less than that observed with IL-12 monotherapy.

4. The method of claim 2 or 3, wherein the mean IL-10 serum trough concentration is at least 2.5 ng/mL.

5. The method of claim 4, wherein the mean IL-10 serum trough concentration is at least 5.0 ng/mL.

6. The method of claim 5, wherein the mean IL-10 serum trough concentration is at least 7.5 ng/mL.

7. The method of claim 6, wherein the mean IL-10 serum trough concentration is at least 10.0 ng/mL.

8. The method of claim 7, wherein the mean IL-10 serum trough concentration is at least 15.0 ng/mL.

9. The method of claim 8, wherein the mean IL-10 serum trough concentration is at least 20.0 ng/mL.

10. The method of claim 3, wherein the period of time is at least 12 hours.

11. The method of claim 10, wherein the period of time is at least 24 hours.

12. The method of claim 11, wherein the period of time is at least 48 hours.

13. The method of claim 12, wherein the period of time is at least 72 hours.

14. The method of claim 13, wherein the period of time is at least 1 week.

15. The method of claim 14, wherein the period of time is at least 2 weeks.

16. The method of claim 15, wherein the period of time is at least 1 month.

17. The method of claim 3, wherein the mean IL-10 serum trough concentration is maintained for at least 95% of the period of time.

18. The method of claim 17, wherein the mean IL-10 serum trough concentration is maintained for at least 98% of the period of time.

19. The method of claim 18, wherein the mean IL-10 serum trough concentration is maintained for 100% of the period of time.

20. The method of any one of claims 1-19, wherein the PEG-IL-10 comprises mature human IL-10.

21. The method of any one of claims 1-19, wherein the PEG-IL-10 comprises a variant of mature human IL-10, and wherein the variant exhibits activity comparable to the activity of mature human IL-10.

22. The method of any one of claims 1-21, wherein the amount of the PEG-IL-10 is from 10.0 μg/kg/day to 20.0 μg/kg/day.

23. The method of any one of claims 1-21, wherein the amount of the PEG-IL-10 is from 11.0 μg/kg/day to 19.0 μg/kg/day.

24. The method ofany one of claims 1-21, wherein the amount of the PEG-IL-10 is from 12.0 μg/kg/day to 18.0 μg/kg/day.

25. The method of any one of claims 1-21, wherein the amount of the PEG-IL-10 is from 13.0 μg/kg/day to 17.0 μg/kg/day.

26. The method of any one of claims 1-21, wherein the amount of the PEG-IL-10 is from 14.0 μg/kg/day to 16.0 μg/kg/day.

27. The method of any one of claims 1-21, wherein the amount of the PEG-IL-10 is about 15.0 μg/kg/day,

28. The method of any one of claims 1-27, wherein the amount of the IL-12 agent is from 0.01 μg/kg/day to 10.0 μg/kg/day.

29. The method of any one of claims 1-27, wherein the amount of the IL-12 agent is from 0.05 μg/kg/day to 9.5 μg/kg/day.

30. The method of any one of claims 1-27, wherein the amount of the IL-12 agent is from 0.1 μg/kg/day to 10.0 μg/kg/day.

31. The method of any one of claims 1-27, wherein the amount of the IL-12 agent is from 0.1 μg/kg/day to 9.0 μg/kg/day.

32. The method of any one of claims 1-27, wherein the amount of the IL-12 agent is from 0.5 μg/kg/day to 8.5 μg/kg/day.

33. The method of any one of claims 1-27, wherein the amount of the IL-12 agent is from 1.0 μg/kg/day to 10.0 μg/kg/day.

34. The method of any one of claims 1-27, wherein the amount of the IL-12 agent is from 1.0 μg/kg/day to 8.0 μg/kg/day.

35. The method of any one of claims 1-27, wherein the amount of the IL-12 agent is from 1.5 μg/kg/day to 7.5 μg/kg/day.

36. The method of any one of claims 1-27, wherein the amount of the IL-12 agent is from 2.0 μg/kg/day to 7.0 μg/kg/day.

37. The method of any one of claims 1-27, wherein the amount of the IL-12 agent is from 2.5 μg/kg/day to 6.5 μg/kg/day.

38. The method of any one of claims 1-27, wherein the amount of the IL-12 agent is from 3.0 μg/kg/day to 6.0 μg/kg/day.

39. The method of any one of claims 1-27, wherein the amount of the IL-12 agent is from 3.5 μg/kg/day to 5.5 μg/kg/day.

40. The method of any one of claims 1-27, wherein the amount of the IL-12 agent is from 4.0 μg/kg/day to 5.0 μg/kg/day.

41. The method of any one of claims 1-27, wherein the amount of the IL-12 agent is about 4.5 μg/kg/day.

42. The method of any one of claims 1-41, wherein the PEG-IL-10 comprises at least one PEG molecule covalently attached to at least one amino acid residue of at least one subunit of IL-10.

43. The method of any one of claims 1-41, wherein the PEG-IL-10 comprises a mixture of mono-pegylated and di-pegylated IL-10.

44. The method of claim 42 or 43, wherein the PEG component of the PEG-IL-10 has a molecular mass from about 5 kDa to about 20 kDa.

45. The method of claim 42 or 43, wherein the PEG component of the PEG-IL-10 has a molecular mass greater than about 20 kDa.

46. The method of claim 42 or 43, wherein the PEG component of the PEG-IL-10 has a molecular mass of at least about 30kD.

47. The method of any one of claims 1-46, wherein the IL-12 agent is mature human IL-12.

48. The method of any one of claims 1-46, wherein the IL-12 agent is a variant of mature human IL-12, and wherein the variant exhibits activity comparable to the activity of mature human IL-12.

49. The method of any one of claims 1-48, wherein the cancer-related disease, disorder or condition is a solid tumor or a lymphoma.

50. The method of claim 49, wherein the solid tumor is selected from the group consisting of breast cancer, prostate cancer, lung cancer, liver cancer, pancreatic cancer, brain cancer, stomach cancer, ovarian cancer, kidney cancer, testicular cancer, and melanoma.

51. The method of any one of claims 1-48, wherein the cancer-related disease, disorder or condition is an immune-insensitive tumor.

52. The method of claim 51, wherein the immune-insensitive tumor is selected from the group consisting of colon, gastroesophageal, pancreatic and breast cancer.

53. The method of any one of claims 1-52, wherein the effects of the PEG-IL-10 and the IL-12 agent are additive.

54. The method of any one of claims 1-52, wherein the effects of the PEG-IL-10 and the IL-12 agent are synergistic.

55. The method of any one of claims 1-54, wherein the PEG-IL-10 is administered to the subject at least twice daily.

56. The method of any one of claims 1-54, wherein the PEG-IL-10 is administered to the subject at least once daily.

57. The method of any one of claims 1-54, wherein the PEG-IL-10 is administered to the subject at least every 72 hours.

58. The method of any one of claims 1-54, wherein the PEG-IL-10 is administered to the subject at least once weekly.

59. The method of any one of claims 1-54, wherein the PEG-IL-10 is administered to the subject at least every 2 weeks.

60. The method of any one of claims 1-54, wherein the PEG-IL-10 is administered to the subject at least once monthly.

61. The method of any one of claims 1-60, further comprising administering at least one additional prophylactic or therapeutic agent.

62. The method of claim 61, wherein the additional prophylactic or therapeutic agent is a chemotherapeutic agent.

63. The method of any one of claims 1-62, wherein the subject is a human.

64. The method of any one of claims 1-63 wherein the administering is by parenteral injection.

65. The method of claim 64, wherein the parenteral injection is subcutaneous.

66. A pharmaceutical composition, comprising an amount of a PEG-IL-10 and an IL-12 agent of any one of claims 1-65, and a pharmaceutically acceptable diluent, carrier or excipient.

67. The pharmaceutical composition of claim 66, wherein the excipient is an isotonic injection solution.

68. The pharmaceutical composition of claim 66, wherein the composition is suitable for human administration.

69. The pharmaceutical composition of any one of claims 66-68, further comprising at least one additional prophylactic or therapeutic agent.

70. A sterile container comprising the pharmaceutical composition of any one of claims 67-69.

71. The sterile container of claim 70, wherein the sterile container is a syringe.

72. A kit comprising the sterile container of claim 70 or 71.

73. The kit of claim 72, further comprising a second sterile container comprising at least one additional prophylactic or therapeutic agent.


SEQUENCE LISTING (24 sequences)

SEQ ID NO: 1 - protein, 306 aa, Homo sapiens
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 2 - protein, 197 aa, Homo sapiens
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 3 - protein, 11 aa, Artificial sequence
  Synthetic polypeptide
YGRKKRRQRRR

SEQ ID NO: 4 - protein, 12 aa, Artificial sequence
  Synthetic polypeptide
RRQRRTSKLMKR

SEQ ID NO: 5 - protein, 27 aa, Artificial sequence
  Synthetic polypeptide
GWTLNSAGYLLGKINLKALAALAKKIL

SEQ ID NO: 6 - protein, 33 aa, Artificial sequence
  Synthetic polypeptide
KALAWEAKLAKALAKALAKHLAKALAKALKCEA

SEQ ID NO: 7 - protein, 16 aa, Artificial sequence
  Synthetic polypeptide
RQIKIWFQNRRMKWKK

SEQ ID NO: 8 - protein, 9 aa, Artificial sequence
  Synthetic polypeptide
RKKRRQRRR

SEQ ID NO: 9 - protein, 8 aa, Artificial sequence
  Synthetic polypeptide
RKKRRQRR

SEQ ID NO: 10 - protein, 11 aa, Artificial sequence
  Synthetic polypeptide
YARAAARQARA

SEQ ID NO: 11 - protein, 11 aa, Artificial sequence
  Synthetic polypeptide
THRLPRRRRRR

SEQ ID NO: 12 - protein, 11 aa, Artificial sequence
  Synthetic polypeptide
GGRRARRRRRR

SEQ ID NO: 13 - protein, 5 aa, Artificial sequence
  Synthetic polypeptide
  MISC_FEATURE (1)..(5): This stretch of residues may be repeated up to 50 times.
GSGGS

SEQ ID NO: 14 - protein, 5 aa, Artificial sequence
  Synthetic polypeptide
  MISC_FEATURE (1)..(1): This residue may be repeated up to 20 times.
  MISC_FEATURE (1)..(5): This stretch of residues may be repeated up to 20 times.
  MISC_FEATURE (2)..(2): This residue may be repeated up to 20 times.
  MISC_FEATURE (3)..(3): This residue may be repeated up to 20 times.
  MISC_FEATURE (4)..(4): This residue may be repeated up to 20 times.
  MISC_FEATURE (5)..(5): This residue may be repeated up to 20 times.
GSGSG

SEQ ID NO: 15 - protein, 5 aa, Artificial sequence
  Synthetic polypeptide
  MISC_FEATURE (1)..(5): This stretch of residues may be repeated up to 20 times.
  MISC_FEATURE (5)..(5): This residue may be repeated up to 20 times.
GSGGS

SEQ ID NO: 16 - protein, 5 aa, Artificial sequence
  Synthetic polypeptide
  MISC_FEATURE (1)..(4): This stretch of residues may be repeated up to 20 times.
  MISC_FEATURE (4)..(4): This residue may be repeated up to 20 times.
GSGSG

SEQ ID NO: 17 - protein, 4 aa, Artificial sequence
  Synthetic polypeptide
  MISC_FEATURE (1)..(4): This stretch of residues may be repeated up to 20 times.
  MISC_FEATURE (4)..(4): This residue may be repeated up to 20 times.
GGGS

SEQ ID NO: 18 - protein, 4 aa, Artificial sequence
  Synthetic polypeptide
GGSG

SEQ ID NO: 19 - protein, 5 aa, Artificial sequence
  Synthetic polypeptide
GGSGG

SEQ ID NO: 20 - protein, 5 aa, Artificial sequence
  Synthetic polypeptide
GSGGG

SEQ ID NO: 21 - protein, 5 aa, Artificial sequence
  Synthetic polypeptide
GGGSG

SEQ ID NO: 22 - protein, 5 aa, Artificial sequence
  Synthetic polypeptide
GSSSG

SEQ ID NO: 23 - protein, 4 aa, Artificial sequence
  Synthetic polypeptide
  MISC_FEATURE (1)..(4): This stretch of residues may be repeated up to 50 times.
GGGS

SEQ ID NO: 24 - protein, 5 aa, Artificial sequence
  Synthetic polypeptide
  MISC_FEATURE (1)..(5): This stretch of residues may be repeated up to 50 times.
GGGGS